(19) United States
(12) Patent Application Publication
(10) Pub. No.: US 2026/0306503 A1
(43) Pub. Date: Oct. 8, 2026

(54) (S)-2-HYDROXY-6-((4-(2-(2-HYDROXYETHYL)NICOTINOYL)MORPHOLIN-3-YL)METHOXY)BENZALDEHYD FOR USE IN THE TREATMENT OF SICKLE CELL DISEASE

(71) Applicant: Global Blood Therapeutics, Inc., South San Francisco, CA (US)

(72) Inventors: Aline BERGESCH BARTH, Collegeville, PA (US); Kenneth Leslie DUCHIN, Delray Beach, FL (US); Eleanor Allen LISBON, Kansas City, MO (US); Arthur Chi Hang LO, San Francisco, CA (US); Eric Iredell Zimmerman, Richmond, VA (US)

(21) Appl. No.: 19/490,232

(22) PCT Filed: Jun. 11, 2024
(86) PCT No.: PCT/IB2024/055707

§ 371 Date: Dec. 4, 2025

Related U.S. Application Data
(60) Provisional application No. 63/507,869, filed on Jun. 13, 2023.
(60) Provisional application No. 63/652,282, filed on May 28, 2024.

(51) Int. Cl. A61K 31/5377; A61K 31/17; A61P 7/06

(52) U.S. Cl. CPC A61K 31/5377; A61K 31/17; A61P 7/06

(57) ABSTRACT

Provided herein are methods for treating sickle cell disease comprising administering Compound I, a modulator of CN hemoglobin, according to certain dosing regimens.

### FIELD

The present disclosure provides for methods for treating sickle cell disease comprising administering Compound I, (S)-2-hydroxy-6-((4-(2-(2-hydroxyethyl)nicotinoyl)morpholin-3-yl)methoxy)benzaldehyde, or a pharmaceutically acceptable salt thereof, a modulator of hemoglobin, according to certain dosing regimens.

### BACKGROUND

Sickle cell disease (SCD) is a disorder of the red blood cells, found particularly among those of African and Mediterranean descent. The basis for SCD is found in sickle hemoglobin (HbS), which contains a point mutation relative to the prevalent peptide sequence of hemoglobin A (HbA).

Hemoglobin (Hb) transports oxygen molecules from the lungs to various tissues and organs throughout the body. Hemoglobin binds and releases oxygen through conformational changes. Sickle hemoglobin (HbS) contains a point mutation where glutamic acid is replaced with valine, making HbS susceptible to polymerization under hypoxic conditions to give the HbS containing red blood cells (RBCs) their characteristic sickle shape. The sickled cells are also more rigid than normal RBCs, and their lack of flexibility can lead to blockage of blood vessels.

Compounds, such as Compound I, that modulate hemoglobin and are useful in treating disorders mediated by abnormal Hb (such as HbS) are disclosed in U.S. Pat. No. 10,683,285, the disclosure of which is hereby incorporated by reference in its entirety.

### SUMMARY

The present disclosure provides for methods for treating sickle cell disease in a subject in need thereof, comprising administering to the subject a first dose of Compound I of formula:

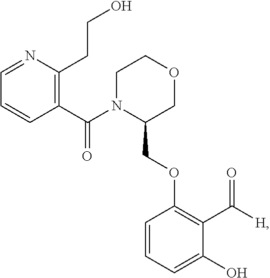

or a pharmaceutically acceptable salt thereof, followed by a second dose of Compound I, or a pharmaceutically acceptable salt thereof.

Accordingly, in a first aspect, the present disclosure provides for a method for treating sickle cell disease in a subject in need thereof, comprising administering to the subject a first dose of Compound I, or a pharmaceutically acceptable salt thereof, for eight or more days, followed thereafter by a second dose of Compound I, or a pharmaceutically acceptable salt thereof, wherein the administrations of the first dose and the second dose provide about 25% to about 60% hemoglobin occupancy of Compound I.

In a second aspect, the present disclosure provides for a method for treating sickle cell disease in a subject in need thereof, comprising administering to the subject a first dose of about 200 mg per day to about 1600 mg per day of Compound I, or a pharmaceutically acceptable salt thereof, for eight or more days, followed thereafter by a second dose of about 25 mg per day to about 500 mg per day of Compound I, or a pharmaceutically acceptable salt thereof.

In a third aspect, the present disclosure provides for a method of treating sickle cell disease in a subject in need thereof, comprising administering to the subject a first dose of about 300 mg once daily (QD) of Compound I, or a pharmaceutically acceptable salt thereof, for fourteen days, followed thereafter by a second dose of about 200 mg once daily (QD) of Compound I, or a pharmaceutically acceptable salt thereof.

### BRIEF DESCRIPTION OF THE DRAWINGS

FIG. 1 shows the Study Schema for Part A of the study described in Example 1.

FIG. 2 shows the Study Schema for Part B of the study described in Example 1.

FIG. 3 shows the Study Schema for Part C of the study described in Example 1.

### DETAILED DESCRIPTION

### 1. Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. In certain embodiments, the term “about” includes the indicated amount±10%. Also, to the term “about X” includes description of “X”.

Recitation of numeric ranges of values throughout the disclosure is intended to serve as a shorthand notation of referring individually to each separate value falling within the range inclusive of the values defining the range, and each separate value is incorporated in the specification as if it were individually recited herein.

Forms of Compound I or salts or solvates thereof are provided herein. In some embodiments, reference to a form of Compound I or a salt or a solvate thereof means that at least 50% to 99% (e.g., at least 50%, at least 55%, at least 60%, at least 65%, at least 70%, at least 75%, at least 80%, at least 85%, at least 90%, at least 95%, or at least 99%) of Compound I or a salt or a solvate thereof present in a composition is in the designated form. For instance, in some embodiments, reference to Compound I Form I means that at least 50%, at least 55%, at least 60%, at least 65%, at least 70%, at least 75%, at least 80%, at least 85%, at least 90%, at least 95%, or at least 99% of Compound I present in a composition is in Form I.

The term “solid form” refers to a type of solid-state material that includes amorphous as well as crystalline forms. The term “crystalline form” refers to polymorphs as well as solvates, etc. The term “polymorph” refers to a particular crystal structure having particular physical properties such as X-ray diffraction, melting point, and the like.

The term “solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

The term “desolvated” refers to a Compound I form that is a solvate as described herein, and from which solvent molecules have been partially or completely removed. Desolvation techniques to produce desolvated forms include, without limitation, exposure of a Compound I form (solvate) to a vacuum, subjecting the solvate to elevated temperature, exposing the solvate to a stream of gas, such as air or nitrogen, or any combination thereof. Thus, a desolvated Compound I form can be anhydrous, i.e., completely without solvent molecules, or partially solvated wherein solvent molecules are present in stoichiometric or non-stoichiometric amounts.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

Any formula or structure given herein, including Compound I, is also intended to represent unlabeled forms as well as isotopically labeled forms of the compounds. It is understood that for any given atom, the isotopes may be present essentially in ratios according to their natural occurrence, or one or more particular atoms may be enhanced with respect to one or more isotopes using synthetic methods known to one skilled in the art. Thus, hydrogen includes for example 1H, 2H, 3H; carbon includes for example 11C, 12C, 13C, 14C; oxygen includes for example 16O, 17O, 18O; nitrogen includes for example 13N, 14N, 15N; sulfur includes for example 32S, 33S, 34S, 35S, 36S, 37S, 38S; fluoro includes for example 17F, 18F, 19F; chloro includes for example 35Cl, 36Cl, 37Cl, 38Cl, 39Cl; and the like.

“Pharmaceutically acceptable” or “physiologically acceptable” refer to forms as described herein, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the forms described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like. In some embodiments, a pharmaceutically acceptable salt does not include a salt of a primary amine.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. A compound described herein, solid form described herein, or a salt or a solvate thereof, may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In some embodiments, the subject is a human.

The term “therapeutically effective amount” or “effective amount” of a compound or solid form described herein, or a salt or a solvate thereof, means an amount sufficient to effect treatment when administered to a subject, to provide a therapeutic benefit such as amelioration of symptoms or slowing of disease progression. For example, a therapeutically effective amount may be an amount sufficient to decrease a symptom of a sickle cell disease. The therapeutically effective amount may vary depending on the subject, and disease or condition being treated, the weight and age of the subject, the severity of the disease or condition, and the manner of administering, which can readily be determined by one of ordinary skill in the art.

In some embodiments, a therapeutically effective amount can also be ascertained by evaluating biomarkers of the disease or disorder to be treated. In the case of SCD, biomarkers may include measuring blood Hb levels, hemolysis (e.g., via blood bilirubin levels), percent Hb occupancy by compound, partial pressure of O2 at which Hb is 20% saturated (p20) or 50% saturated (p50) with O2, and RBC deformability as a function of oxygen tension (pO2) including determination of maximum RBC deformability under normoxic conditions (EImax), minimum RBC deformability under hypoxic conditions (EImin), and the specific pO2 level at which RBC sickling occurs, i.e., the point of sickling (PoS). Therapeutic efficacy in SCD may also include measuring oxygen deliver to tissues including, e.g., microvasculature oxygen saturation, cerebral blood flow, oxygen extraction fraction, and cerebral metabolic rate for oxygen, which may be measured by, e.g., diffuse correlation spectroscopy (DSC) and/or frequency-domain near-infrared spectroscopy (FDNIRS).

In some embodiments, the therapeutically effective amount is determined by measuring the percent of hemoglobin occupied by the compound, i.e., the percent Hb occupancy. The percent Hb occupancy is a measure of target engagement defined as the percentage of hemoglobin bound by a compound (e.g., Compound I) calculated as the molar concentration of the compound (e.g., Compound I) in RBCs divided by the molar concentration of hemoglobin in RBCs (the mean corpuscular hemoglobin concentration calculated from the hematology panel). The values for concentration of a compound (e.g., Compound I) in RBCs is calculated from the concentrations in whole blood and plasma, and the hematocrit.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the forms described herein and compositions described herein may be used for a variety of purposes, including therapeutic and experimental purposes. For example, the forms described herein and compositions described herein may be used ex vivo to determine the optimal schedule and/or dosing of administration of a form of the present disclosure for a given indication, cell type, individual, and other parameters. Information gleaned from such use may be used for experimental purposes or in the clinic to set protocols for in vivo treatment. Other ex vivo uses for which the forms described herein and compositions described herein may be suited are described below or will become apparent to those skilled in the art. The selected forms described herein may be further characterized to examine the safety or tolerance dosage in human or non-human subjects. Such properties may be examined using commonly known methods to those skilled in the art.

The term “hemoglobin” as used herein refers to any hemoglobin protein, including normal hemoglobin (HbA) and abnormal hemoglobin, such as sickle hemoglobin (HbS).

The term “sickle cell disease” refers to diseases mediated by sickle hemoglobin (HbS) that results from a single point mutation in the hemoglobin (Hb). Sickle cell diseases include sickle cell anemia (HbSS), hemoglobin SC disease (HbSC), hemoglobin S beta-plus-thalassemia (HbS/β+) and hemoglobin S beta-zero-thalassemia (HbS/β0).

“First dose” refers to an initial dose of a compound as described herein, or series of such doses, given to achieve a target therapeutic level in the subject in a desired amount of time. In some embodiments, a “first dose” refers to a “loading dose”. In some embodiments, a “loading dose” refers to a “first dose”.

“Second dose” refers to a dose of a compound as described herein, or series of such doses, administered to maintain a desired therapeutic level of the compound in the subject. In some embodiments, a “second dose” refers to a “maintenance dose”. In some embodiments, a “maintenance dose” refers to a “second dose”.

A target therapeutic level can be assessed based on the percent hemoglobin occupancy or on parameters related thereto such as concentration of Compound I in the subject's blood relative to the subject's hematocrit.

### 2. Compound I and Methods of Use Thereof

Provided herein are methods for treating sickle cell disease (SCD). Sickle hemoglobin (HbS) contains a point mutation where glutamic acid is replaced with valine, making HbS susceptible to polymerization under hypoxic conditions to give the HbS containing red blood cells their characteristic sickle shape. The sickled cells are also more rigid than normal red blood cells, and their lack of flexibility can lead to blockage of blood vessels. It is contemplated that an approach to therapy would be to maintain the HbS in the oxygenated state, as polymerization occurs only in the deoxygenated state under hypoxic conditions.

Provided herein are methods for treating sickle cell disease (SCD) comprising administering (S)-2-hydroxy-6-((4-(2-(2-hydroxyethyl)nicotinoyl)morpholin-3-yl)methoxy)benzaldehyde (Compound I) or a pharmaceutically acceptable salt thereof. Compound I has the following formula:

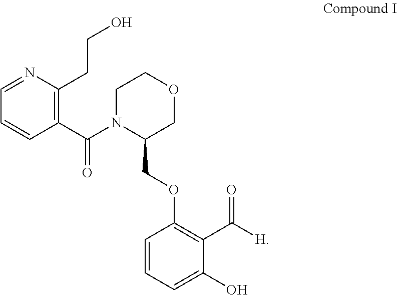

Compound I is a modulator of hemoglobin, and its synthesis and method of use thereof is described in U.S. Pat. No. 10,683,285.

In some embodiments, Compound I is a solid form.

In some embodiments, Compound I is a crystalline form. In some embodiments, Compound I is a crystalline form characterized by an X-ray powder diffractogram comprising the following peaks: 18.3, 23.4, and 26.1°2θ±0.2°2θ (Compound I Form I), as determined on a diffractometer using Cu-Kα radiation. In some embodiments, the diffractogram further comprises one or more peaks at: 10.8 or 17.3°2θ±0.2°2θ. In some embodiments, the crystalline form is characterized by a differential scanning calorimetry (DSC) curve that comprises an endotherm at about 111° C. (onset temperature).

Amorphous Compound I may be made according to methods known in the art. A solution of amorphous Compound I in acetonitrile (>540 mg/mL) was refrigerated for 4 days and then placed in a freezer for 1 day. The solids were filtered and dried under nitrogen to provide Compound I Form I.

Compound I Form I was also prepared as follows: Amorphous Compound I was slurried in ether with seeding with Compound I Form I from another experiment (prepared as described herein) at ambient temperature for 1 day, providing Compound I Form I.

X-ray Powder Diffraction (XRPD): XRPD figures were generated using SSCI Pattern Match 3.0.4, unvalidated software. XRPD patterns were collected with a PANalytical X'Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e or NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X'Celerator) located 240 mm from the specimen and Data Collector software v. 2.2b or v. 5.5.

Differential Scanning Calorimetry (DSC): DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan was then inserted into the DSC cell. A weighed aluminum pan configured as the sample pan was placed on the reference side of the cell. The pan lid was pierced prior to sample analysis. Samples were analyzed from −30° C. to 250° C.@10°/min.

Some embodiments provided herein comprise methods of administering Compound I or a pharmaceutically acceptable salt thereof. Some embodiments provided herein comprise methods of administering Compound I. Some embodiments provided herein comprise methods of administering a pharmaceutically acceptable salt of Compound I. In some embodiments, the dose of a pharmaceutically acceptable salt of Compound I is adjusted to be equivalent to the dose of Compound I.

Some embodiments provide for methods for treating sickle cell disease by administration of Compound I, or a pharmaceutically acceptable salt thereof, wherein the administration provides about 25% to about 80% hemoglobin occupancy. Some embodiments provide for methods for treating sickle cell disease by administration of Compound I, or a pharmaceutically acceptable salt thereof, wherein the administration provides about 20% to about 60% hemoglobin occupancy.

Some embodiments provide for methods for treating sickle cell disease by administration of Compound I, or a pharmaceutically acceptable salt thereof, wherein the administration provides about 30% to about 50% hemoglobin occupancy.

It is contemplated that the methods described herein can achieve targeted hemoglobin occupancy and attain desired hematological effects, therefore improving clinical outcomes (such as numerically fewer vaso-occlusive crises) for individuals living with SCD.

In one aspect, the present disclosure provides for methods for treating sickle cell disease in a subject in need thereof, comprising administering to the subject a first dose of Compound I of formula:

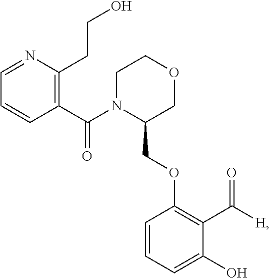

or a pharmaceutically acceptable salt thereof, for eight or more days, followed by a second dose of Compound I, or a pharmaceutically acceptable salt thereof, thereafter, wherein the administrations of the first dose and the second dose provide about 25% to about 80% hemoglobin occupancy by Compound I.

In another aspect, the present disclosure provides for methods for treating sickle cell disease in a subject in need thereof, comprising administering to the subject a first dose of Compound I of formula:

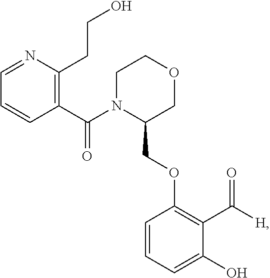

or a pharmaceutically acceptable salt thereof, for eight or more days, followed by a second dose of Compound I, or a pharmaceutically acceptable salt thereof, thereafter, wherein the administrations of the first dose and the second dose provide about 25% to about 60% hemoglobin occupancy by Compound I.

In another aspect, the present disclosure provides for methods for treating sickle cell disease in a subject in need thereof, comprising administering to the subject a first dose of Compound I of formula:

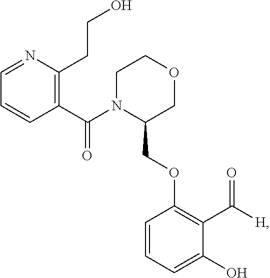

or a pharmaceutically acceptable salt thereof, for eight or more days, followed by a second dose of Compound I, or a pharmaceutically acceptable salt thereof, wherein the administration of the first dose and the second dose provide about 30% to about 50% hemoglobin occupancy by Compound I.

In some embodiments, the percent hemoglobin occupancy, as provided by the administration of Compound I, is about 25% to about 80%. In some embodiments, the percent hemoglobin occupancy, as provided by the administration of Compound I, is about 25% to about 75%. In some embodiments, the percent hemoglobin occupancy, as provided by the administration of Compound I, is about 25% to about 70%. In some embodiments, the percent hemoglobin occupancy, as provided by the administration of Compound I, is about 25% to about 65%. In some embodiments, the percent hemoglobin occupancy, as provided by the administration of Compound I, is about 25% to about 60%. In some embodiments, the percent hemoglobin occupancy is about 25% to about 55%.

In some embodiments, the percent hemoglobin occupancy (as provided by the administration of Compound I) is about 25% to about 50%. In some embodiments, the percent hemoglobin occupancy is about 25% to about 45%. In some embodiments, the percent hemoglobin occupancy is about 30% to about 60%. In some embodiments, the percent hemoglobin occupancy is about 30% to about 55%. In some embodiments, the percent hemoglobin occupancy is about 30% to about 50%. In some embodiments, the percent hemoglobin occupancy is about 30% to about 45%. In some embodiments, the percent hemoglobin occupancy is about 30% to about 40%. In some embodiments, the percent hemoglobin occupancy is about 30% to about 35%. In some embodiments, the percent hemoglobin occupancy is about 35% to about 60%. In some embodiments, the percent hemoglobin occupancy is about 35% to about 55%. In some embodiments, the percent hemoglobin occupancy is about 35% to about 50%. In some embodiments, the percent hemoglobin occupancy is about 35% to about 40%. In some embodiments, the percent hemoglobin occupancy is about 40% to about 60%. In some embodiments, the percent hemoglobin occupancy is about 40% to about 55%. In some embodiments, the percent hemoglobin occupancy is about 40% to about 50%. In some embodiments, the percent hemoglobin occupancy is about 40% to about 45%.

In some embodiments, the percent hemoglobin occupancy may be calculated as described herein. In some embodiments, percent hemoglobin occupancy is the molar ratio of Compound I concentration to the Hb concentration in red blood cells.

In some embodiments, the present disclosure provides methods for treating sickle cell disease in a subject in need thereof, comprising administering to the subject a first dose of Compound I, or a pharmaceutically acceptable salt thereof, for eight or more days to achieve a target therapeutic level in the subject, followed by a second dose of Compound I, or a pharmaceutically acceptable salt thereof, thereafter to maintain the target therapeutic level in the subject, wherein on a daily dose basis the first dose is greater than the second dose.

In some embodiments, the present disclosure provides methods for treating sickle cell disease in a subject in need thereof, comprising administering to the subject a first dose of Compound I, or a pharmaceutically acceptable salt thereof, for eight or more days to achieve a target therapeutic level in the subject, followed thereafter by a second dose of Compound I, or a pharmaceutically acceptable salt thereof, to maintain the target therapeutic level in the subject, wherein on a daily dose basis the first dose is greater than the second dose (i.e. the first dose per day is greater than the second dose per day).

In some embodiments, the ratio of first dose to second dose is about 88:1 to about 2:1. In some embodiments, the ratio of first dose to second dose is about 60:1 to about 2:1. In some embodiments, the ratio of first dose to second dose is about 40:1 to about 2:1. In some embodiments, the ratio of first dose to second dose is about 20:1 to about 2:1. In some embodiments, the ratio of first dose to second dose is about to second dose 10:1 to about 2:1.

In some embodiments, the ratio of first dose per day to second dose per day is about 88:1 to about 1:1. In some embodiments, the ratio of first dose per day to second dose per day is about 60:1 to about 1:1. In some embodiments, the ratio of first dose per day to second dose per day is about 40:1 to about 1:1. In some embodiments, the ratio of first dose per day to second dose per day is about 20:1 to about 1:1. In some embodiments, the ratio of first dose per day to second dose per day is about 10:1 to about 1:1. In some embodiments, the ratio of first dose per day to second dose per day is about 4:1 to about 1:1. In some embodiments, the ratio of first dose per day to second dose per day is about 2:1 to 1:1.

Some embodiments provide for methods for treating sickle cell disease in a subject in need thereof, comprising administering to the subject a first dose of about 100 mg per day to about 2200 mg per day of Compound I, or a pharmaceutically acceptable salt thereof, for eight or more days, followed thereafter by a second dose of about 25 mg per day to about 500 mg per day of Compound I, or a pharmaceutically acceptable salt thereof.

Some embodiments provide for methods for treating sickle cell disease in a subject in need thereof, comprising administering to the subject a first dose of about 200 mg per day to about 1600 mg per day of Compound I, or a pharmaceutically acceptable salt thereof, for eight or more days, followed thereafter by a second dose of about 25 mg per day to about 500 mg per day of Compound I, or a pharmaceutically acceptable salt thereof.

Some embodiments provide for methods for treating sickle cell disease in a subject in need thereof, comprising administering to the subject a first dose of about 300 mg per day to about 1500 mg per day of Compound I, or a pharmaceutically acceptable salt thereof, for eight or more days, followed thereafter by a second dose of about 25 mg per day to about 500 mg per day of Compound I, or a pharmaceutically acceptable salt thereof.

Some embodiments provide for methods for treating sickle cell disease in a subject in need thereof, comprising administering to the subject a first dose of about 300 mg per day to about 1500 mg per day of Compound I, or a pharmaceutically acceptable salt thereof, for eight or more days, followed thereafter by a second dose of about 25 mg per day to about 250 mg per day of Compound I, or a pharmaceutically acceptable salt thereof.

Some embodiments provide for methods for treating sickle cell disease in a subject in need thereof, comprising administering to the subject a first dose of about 300 mg per day to about 900 mg per day of Compound I, or a pharmaceutically acceptable salt thereof, for eight or more days, followed thereafter by a second dose of about 25 mg per day to about 250 mg per day of Compound I, or a pharmaceutically acceptable salt thereof.

Some embodiments provide for methods for treating sickle cell disease in a subject in need thereof, comprising administering to the subject a first dose of about 200 mg per day to about 900 mg per day of Compound I, or a pharmaceutically acceptable salt thereof, for eight or more days, followed by a second dose of about 25 mg per day to about 250 mg per day of Compound I, or a pharmaceutically acceptable salt thereof, thereafter.

Some embodiments provide for methods for treating sickle cell disease in a subject in need thereof, comprising administering to the subject a first dose of about 200 mg per day to about 900 mg per day of Compound I, or a pharmaceutically acceptable salt thereof, for eight or more days, followed thereafter by a second dose of about 25 mg per day to about 200 mg per day of Compound I, or a pharmaceutically acceptable salt thereof.

Some embodiments provide for methods for treating sickle cell disease in a subject in need thereof, comprising administering to the subject a first dose of about 200 mg per day to about 900 mg per day of Compound I, or a pharmaceutically acceptable salt thereof, for eight or more days, followed thereafter by a second dose of about 25 mg per day to about 150 mg per day of Compound I, or a pharmaceutically acceptable salt thereof.

Some embodiments provide for methods for treating sickle cell disease in a subject in need thereof, comprising administering to the subject a first dose of about 300 mg per day to about 900 mg per day of Compound I, or a pharmaceutically acceptable salt thereof, for fourteen days, followed by a second dose of about 50 mg per day to about 250 mg per day of Compound I, or a pharmaceutically acceptable salt thereof.

In some embodiments, the first dose comprises about 100 mg per day to about 2200 mg per day of Compound I, or a pharmaceutically acceptable salt thereof. In some embodiments, the first dose comprises about 200 mg per day to about 2000 mg per day of Compound I, or a pharmaceutically acceptable salt thereof. In some embodiments, the first dose comprises about 200 mg per day to about 1800 mg per day of Compound I, or a pharmaceutically acceptable salt thereof.

In some embodiments, the first dose comprises about 200 mg per day to about 1600 mg per day of Compound I, or a pharmaceutically acceptable salt thereof. In some embodiments, the first dose comprises about 300 mg per day to about 1500 mg per day of Compound I, or a pharmaceutically acceptable salt thereof. In some embodiments, the first dose comprises about 200 mg per day to about 1500 mg per day of Compound I, or a pharmaceutically acceptable salt thereof. In some embodiments, the first dose comprises about 200 mg per day to about 1000 mg per day of Compound I, or a pharmaceutically acceptable salt thereof. In some embodiments, the first dose comprises about 200 mg per day to about 900 mg per day of Compound I, or a pharmaceutically acceptable salt thereof.

In some embodiments, the first dose comprises about 1500 mg per day of Compound I, or a pharmaceutically acceptable salt thereof. In some embodiments, the first dose comprises about 1400 mg per day of Compound I, or a pharmaceutically acceptable salt thereof. In some embodiments, the first dose comprises about 1300 mg per day of Compound I, or a pharmaceutically acceptable salt thereof. In some embodiments, the first dose comprises about 1200 mg per day of Compound I, or a pharmaceutically acceptable salt thereof. In some embodiments, the first dose comprises about 1100 mg per day of Compound I, or a pharmaceutically acceptable salt thereof. In some embodiments, the first dose comprises about 1000 mg per day of Compound I, or a pharmaceutically acceptable salt thereof. In some embodiments, the first dose comprises about 900 mg per day of Compound I, or a pharmaceutically acceptable salt thereof. In some embodiments, the first dose comprises about 800 mg per day of Compound I, or a pharmaceutically acceptable salt thereof. In some embodiments, the first dose comprises about 700 mg per day of Compound I, or a pharmaceutically acceptable salt thereof. In some embodiments, the first dose comprises about 600 mg per day of Compound I, or a pharmaceutically acceptable salt thereof. In some embodiments, the first dose comprises about 500 mg per day of Compound I, or a pharmaceutically acceptable salt thereof. In some embodiments, the first dose comprises about 400 mg per day of Compound I, or a pharmaceutically acceptable salt thereof. In some embodiments, the first dose comprises about 300 mg per day of Compound I, or a pharmaceutically acceptable salt thereof. In some embodiments, the first dose comprises about 200 mg per day of Compound I, or a pharmaceutically acceptable salt thereof. In some embodiments, the first dose comprises about 100 mg per day of Compound I, or a pharmaceutically acceptable salt thereof.

In some embodiments, the first dose is administered for eight to fourteen days. In some embodiments, the first dose is administered for eight to thirteen days. In some embodiments, the first dose is administered for eight to twelve days. In some embodiments, the first dose is administered for eight to eleven days. In some embodiments, the first dose is administered for eight to ten days. In some embodiments, the first dose is administered for eight to nine days.

In some embodiments, the first dose is administered for eight days. In some embodiments, the first dose is administered for nine days. In some embodiments, the first dose is administered for ten days. In some embodiments, the first dose is administered for eleven days. In some embodiments, the first dose is administered for twelve days. In some embodiments, the first dose is administered for thirteen days. In some embodiments, the first dose is administered for fourteen days.

In some embodiments of the methods described herein, the method comprises administering a first dose of about 300 mg once daily (QD) of Compound I, or a pharmaceutically acceptable salt thereof, for eight or more days. In some embodiments, the method comprises administering a first dose of about 300 mg once daily (QD) of Compound I, or a pharmaceutically acceptable salt thereof, for fourteen days.

Following a first dose administered over eight or more days to achieve a target therapeutic level of Compound I, or a pharmaceutically acceptable salt thereof, in the subject, a second dose of Compound I, or a pharmaceutically acceptable salt thereof, is administered thereafter to maintain the target therapeutic level in the subject. In some embodiments, the therapeutic level of Compound I is the determined by measuring the percent hemoglobin occupied, or % Hb occupancy, by Compound I.

In some embodiments, the second dose is about 25 mg per day to about 600 mg per day of Compound I, or a pharmaceutically acceptable salt thereof. In some embodiments, the second dose is about 25 mg per day to about 550 mg per day of Compound I, or a pharmaceutically acceptable salt thereof. In some embodiments, the second dose is about 25 mg per day to about 500 mg per day of Compound I, or a pharmaceutically acceptable salt thereof. In some embodiments, the second dose is about 25 mg per day to about 450 mg per day of Compound I, or a pharmaceutically acceptable salt thereof. In some embodiments, the second dose is about 25 mg per day to about 400 mg per day of Compound I, or a pharmaceutically acceptable salt thereof. In some embodiments, the second dose is about 25 mg per day to about 350 mg per day of Compound I, or a pharmaceutically acceptable salt thereof. In some embodiments, the second dose is about 25 mg per day to about 300 mg per day of Compound I, or a pharmaceutically acceptable salt thereof.

In some embodiments, the second dose is about 25 mg per day of Compound I, or a pharmaceutically acceptable salt thereof. In some embodiments, the second dose is about 50 mg per day of Compound I, or a pharmaceutically acceptable salt thereof. In some embodiments, the second dose is about 75 mg per day of Compound I, or a pharmaceutically acceptable salt thereof. In some embodiments, the second dose is about 100 mg per day of Compound I, or a pharmaceutically acceptable salt thereof. In some embodiments, the second dose is about 125 mg per day of Compound I, or a pharmaceutically acceptable salt thereof. In some embodiments, the second dose is about 150 mg per day of Compound I, or a pharmaceutically acceptable salt thereof. In some embodiments, the second dose is about 175 mg per day of Compound I, or a pharmaceutically acceptable salt thereof. In some embodiments, the second dose is about 200 mg per day of Compound I, or a pharmaceutically acceptable salt thereof. In some embodiments, the second dose is about 225 mg per day of Compound I, or a pharmaceutically acceptable salt thereof. In some embodiments, the second dose is about 250 mg per day of Compound I, or a pharmaceutically acceptable salt thereof. In some embodiments, the second dose is about 275 mg per day of Compound I, or a pharmaceutically acceptable salt thereof. In some embodiments, the second dose is about 300 mg per day of Compound I, or a pharmaceutically acceptable salt thereof. In some embodiments, the second dose is about 350 mg per day of Compound I, or a pharmaceutically acceptable salt thereof. In some embodiments, the second dose is about 400 mg per day of Compound I, or a pharmaceutically acceptable salt thereof. In some embodiments, the second dose is about 450 mg per day of Compound I, or a pharmaceutically acceptable salt thereof. In some embodiments, the second dose is about 500 mg per day of Compound I, or a pharmaceutically acceptable salt thereof.

In some embodiments, the second dose is administered once daily (QD).

In some embodiments, the first dose is administered for fourteen days. In some embodiments, the first dose is administered for thirteen days. In some embodiments, the first dose is administered for twelve days. In some embodiments, the first dose is administered for eleven days. In some embodiments, the first dose is administered for ten days. In some embodiments, the first dose is administered for nine days.

In some embodiments, the first dose of Compound I, or a pharmaceutically acceptable salt thereof, is administered once daily (QD). In some embodiments, the first dose of Compound I, or a pharmaceutically acceptable salt thereof, is administered twice daily (BID). In some embodiments, the first dose of Compound I, or a pharmaceutically acceptable salt thereof, is administered three times daily (TID). In some embodiments, the first dose of Compound I, or a pharmaceutically acceptable salt thereof, is administered four times daily (QID). In some embodiments, the first dose of Compound I, or a pharmaceutically acceptable salt thereof, is administered every six hours (Q6H).

In some embodiments, the second dose of Compound I, or a pharmaceutically acceptable salt thereof, is administered once daily (QD). In some embodiments, the second dose of Compound I, or a pharmaceutically acceptable salt thereof, is administered twice daily (BID). In some embodiments, the second dose of Compound I, or a pharmaceutically acceptable salt thereof, is administered three times daily (TID). In some embodiments, the second dose of Compound I, or a pharmaceutically acceptable salt thereof, is administered four times daily (QID). In some embodiments, the second dose of Compound I, or a pharmaceutically acceptable salt thereof, is administered every six hours (Q6H).

In some embodiments, the first dose of Compound I, or a pharmaceutically acceptable salt thereof, is administered once daily (QD), and the second dose of Compound I, or a pharmaceutically acceptable salt thereof, is administered once daily (QD) thereafter.

In embodiments disclosed herein, the second dose is administered for as long as the patient requires or benefits from the therapeutic effect provided by such administration.

In some embodiments, the second dose is administered for one to four weeks. In some embodiments, the second dose is administered for one to five weeks. In some embodiments, the second dose is administered for one to six weeks. In some embodiments, the second dose is administered for one to seven weeks. In some embodiments, the second dose is administered for one to eight weeks. In some embodiments, the second dose is administered for one to nine weeks. In some embodiments, the second dose is administered for one to ten weeks. In some embodiments, the second dose is administered for one to twenty weeks. In some embodiments, the second dose is administered for one to thirty weeks. In some embodiments, the second dose is administered for one to forty weeks. In some embodiments, the second dose is administered for one to fifty weeks. In some embodiments, the second dose is administered for one to sixty weeks. In some embodiments, the second dose is administered for one to seventy weeks. In some embodiments, the second dose is administered for one to eighty weeks. In some embodiments, the second dose is administered for one to ninety weeks. In some embodiments, the second dose is administered for one to one hundred weeks.

In some embodiments, the second dose is administered for at least one week. In some embodiments, the second dose is administered for at least two weeks. In some embodiments, the second dose is administered for at least three weeks. In some embodiments, the second dose is administered for at least four weeks. In some embodiments, the second dose is administered for at least five weeks. In some embodiments, the second dose is administered for at least six weeks. In some embodiments, the second dose is administered for at least seven weeks. In some embodiments, the second dose is administered for at least eight weeks. In some embodiments, the second dose is administered for at least nine weeks. In some embodiments, the second dose is administered for at least ten weeks. In some embodiments, the second dose is administered for at least fifteen weeks. In some embodiments, the second dose is administered for at least twenty weeks. In some embodiments, the second dose is administered for at least thirty weeks. In some embodiments, the second dose is administered for at least forty weeks. In some embodiments, the second dose is administered for at least fifty weeks. In some embodiments, the second dose is administered for at least sixty weeks. In some embodiments, the second dose is administered for at least seventy weeks. In some embodiments, the second dose is administered for at least eight weeks. In some embodiments, the second dose is administered for at least ninety weeks. In some embodiments, the second dose is administered for at least one hundred weeks.

In some embodiments, the second dose is administered for at least one year. In some embodiments, the second dose is administered for at least two years. In some embodiments, the second dose is administered for at least three years. In some embodiments, the second dose is administered for at least four years. In some embodiments, the second dose is administered for at least five years. In some embodiments, the second dose is administered for at least six years. In some embodiments, the second dose is administered for at least seven years. In some embodiments, the second dose is administered for at least eight years. In some embodiments, the second dose is administered for at least nine years. In some embodiments, the second dose is administered for at least ten years.

In some embodiments, the second dose is administered for the lifetime of the subject. In some embodiments, the second dose is administered until the subject dies.

In some embodiments, the first dose and second dose are administered consecutively. For example, in some embodiments, the first dose is administered consecutively over fourteen days, and the second dose is first administered on the fifth day.

In some embodiments, the percent hemoglobin occupancy in the subject, as provided by the administration of Compound I, or a pharmaceutically acceptable salt thereof, reaches about 25% to about 60% after about 8 hours to about 24 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 60% after 8 hours to about 20 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 60% after 8 hours to about 16 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 60% after 8 hours to about 12 hours following the completion of the first dose (or loading dose) of Compound I, or a pharmaceutically acceptable salt thereof.

In some embodiments, the percent hemoglobin occupancy in the subject, as provided by the administration of Compound I, or a pharmaceutically acceptable salt thereof, reaches about 25% to about 55% after about 8 hours to about 24 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 50% after about 8 hours to about 24 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 45% after about 8 hours to about 24 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 40% after about 8 hours to about 24 hours following the completion of the first dose (or loading dose) of Compound I, or a pharmaceutically acceptable salt thereof. In some embodiments, the percent hemoglobin occupancy in the subject, as provided by the administration of Compound I, or a pharmaceutically acceptable salt thereof, reaches about 25% to about 35% after about 8 hours to about 24 hours following the completion of the first dose (or loading dose) of Compound I, or a pharmaceutically acceptable salt thereof.

In some embodiments, the percent hemoglobin occupancy in the subject, as provided by the administration of Compound I, or a pharmaceutically acceptable salt thereof, reaches about 25% to about 60% following a loading dose regimen as described herein. For example, in some embodiments, the percent hemoglobin occupancy in the subject, as provided by administration of Compound I, or a pharmaceutically acceptable salt thereof, reaches about 25% to about 60% following a loading dose regimen that comprises administration of 300 mg of Compound I, or a pharmaceutically acceptable salt thereof, once daily (QD) for fourteen days.

In some embodiments, the hemoglobin occupancy in the subject, as provided by the administration of Compound I, or a pharmaceutically acceptable salt thereof, reaches about 25% to about 60% prior to administration of the second dose (or maintenance dose) of Compound I, or a pharmaceutically acceptable salt thereof. For example, in some embodiments, the percent hemoglobin occupancy in the subject, as provided by the administration of Compound I, or a pharmaceutically acceptable salt thereof, reaches about 25% to about 60% following a first dose (loading dose) regimen that comprises administration of 300 mg of Compound I, or a pharmaceutically acceptable salt thereof, once daily for fourteen days, and prior to administration of the second dose (or maintenance dose) of Compound I, or a pharmaceutically acceptable salt thereof.

In some embodiments, the administration of Compound I, or a pharmaceutically acceptable salt thereof, provides a hematocrit value of about 20% to about 50% in the subject.

In some embodiment, the administration of Compound I, or a pharmaceutically acceptable salt thereof, provides a hematocrit value of at least about 20% in the subject. In some embodiment, the administration of Compound I, or a pharmaceutically acceptable salt thereof, provides a hematocrit value of at least about 30% in the subject. In some embodiment, the administration of Compound I, or a pharmaceutically acceptable salt thereof, provides a hematocrit value of about 40% in the subject. In some embodiment, the administration of Compound I, or a pharmaceutically acceptable salt thereof, provides a hematocrit value of about 45% in the subject.

In some embodiments, the administration of Compound I, or a pharmaceutically acceptable salt thereof, provides a blood concentration of Compound I of about 50 μg/mL to about 800 μg/mL. In some embodiments, the administration of Compound I, or a pharmaceutically acceptable salt thereof, provides a blood concentration of Compound I of about 95 μg/mL to about 775 μg/mL. In some embodiments, the administration of Compound I, or a pharmaceutically acceptable salt thereof, provides a blood concentration of Compound I of about 95 μg/mL to about 750 μg/mL. In some embodiments, the administration of Compound I, or a pharmaceutically acceptable salt thereof, provides a blood concentration of Compound I of about 140 μg/mL to about 700 μg/mL. In some embodiments, the administration of Compound I, or a pharmaceutically acceptable salt thereof, provides a blood concentration of Compound I of about 190 μg/mL to about 650 μg/mL. In some embodiments, the administration of Compound I, or a pharmaceutically acceptable salt thereof, provides a blood concentration of Compound I of about 250 μg/mL to about 475 μg/mL. In some embodiments, the administration of Compound I, or a pharmaceutically acceptable salt thereof, provides a blood concentration of Compound I of about 300 μg/mL to about 450 μg/mL. In some embodiments, the administration of Compound I, or a pharmaceutically acceptable salt thereof, provides a blood concentration of Compound I of about 300 μg/mL to about 475 μg/mL.

In some embodiments, the administration of Compound I, or a pharmaceutically acceptable salt thereof, provides an AUC0-24 of Compound I of about 1,200 μg*h/mL to about 19,200 μg*h/mL. In some embodiments, the administration of Compound I, or a pharmaceutically acceptable salt thereof, provides an AUC0-24 of Compound I of about 2,280 μg*h/mL to about 18,600 μg*h/mL. In some embodiments, the administration of Compound I, or a pharmaceutically acceptable salt thereof, provides an AUC0-24 of Compound I of about 2,280 μg*h/mL to about 18,000 μg*h/mL. In some embodiments, the administration of Compound I, or a pharmaceutically acceptable salt thereof, provides an AUC0-24 of Compound I of about 3,360 μg*h/mL to about 16,800 μg*h/mL. In some embodiments, the administration of Compound I, or a pharmaceutically acceptable salt thereof, provides an AUC0-24 of Compound I of about 4,560 μg*h/mL to about 15,600 μg*h/mL. In some embodiments, the administration of Compound I, or a pharmaceutically acceptable salt thereof, provides an AUC0-24 of Compound I of about 6,000 μg*h/mL to about 11,400 μg*h/mL. In some embodiments, the administration of Compound I, or a pharmaceutically acceptable salt thereof, provides an AUC0-24 of Compound I of about 7,200 μg*h/mL to about 10,800 μg*h/mL. In some embodiments, the administration of Compound I, or a pharmaceutically acceptable salt thereof, provides an AUC0-24 of Compound I of about 7,200 μg*h/mL to about 11,400 μg*h/mL.

In some embodiments, the administration of Compound I, or a pharmaceutically acceptable salt thereof, provides an AUC0-24 of Compound I of more than about 1,200 μg*h/mL. In some embodiments, the administration of Compound I, or a pharmaceutically acceptable salt thereof, provides an AUC0-24 of Compound I of more than about 2,280 μg*h/mL. In some embodiments, the administration of Compound I, or a pharmaceutically acceptable salt thereof, provides an AUC0-24 of Compound I of more than about 3,360 μg*h/mL. In some embodiments, the administration of Compound I, or a pharmaceutically acceptable salt thereof, provides an AUC0-24 of Compound I of more than about 4,560 μg*h/mL. In some embodiments, the administration of Compound I, or a pharmaceutically acceptable salt thereof, provides an AUC0-24 of Compound I of more than about 6,000 μg*h/mL. In some embodiments, the administration of Compound I, or a pharmaceutically acceptable salt thereof, provides an AUC0-24 of Compound I of more than about 7,200 μg*h/mL. In some embodiments, the administration of Compound I, or a pharmaceutically acceptable salt thereof, provides an AUC0-24 of Compound I of more than about 10,800 μg*h/mL. In some embodiments, the administration of Compound I, or a pharmaceutically acceptable salt thereof, provides an AUC0-24 of Compound I of more than about 11,400 μg*h/mL. In some embodiments, the administration of Compound I, or a pharmaceutically acceptable salt thereof, provides an AUC0-24 of Compound I of more than about 15,600 μg*h/mL. In some embodiments, the administration of Compound I, or a pharmaceutically acceptable salt thereof, provides an AUC0-24 of Compound I of more than about 16,800 μg*h/mL. In some embodiments, the administration of Compound I, or a pharmaceutically acceptable salt thereof, provides an AUC0-24 of Compound I of more than about 18,000 μg*h/mL. In some embodiments, the administration of Compound I, or a pharmaceutically acceptable salt thereof, provides an AUC0-24 of Compound I of more than about 18,600 μg*h/mL. In some embodiments, the administration of Compound I, or a pharmaceutically acceptable salt thereof, provides an AUC0-24 of Compound I of more than about 19,000 μg*h/mL.

In some embodiments, Compound I, or a pharmaceutically acceptable salt thereof, is administered as a pharmaceutical composition. In some embodiments, Compound I is administered as a pharmaceutical composition.

In some embodiments, Compound I, or a pharmaceutically acceptable salt thereof, is administered orally.

Some embodiments provided herein further comprise administering an additional therapeutic agent.

In some embodiments, the additional therapeutic agent is a modulator of hemoglobin. In some embodiments, the additional therapeutic agent is useful for treating sickle cell disease. In some embodiments, the additional therapeutic agent is useful for treating a complication of sickle cell disease. Non-limiting examples of a complication of sickle cell disease include iron overload, pain, infections, acute chest syndrome, stroke, and pulmonary hypertension. In some embodiments, the additional therapeutic agent is hydroxyurea, L-glutamine, crizanlizumab, or deferiprone. In some embodiments, the additional therapeutic agent is hydroxyurea.

It is to be understood that the method of treatment embodiments of the present disclosure can be presented in a use type format.

Accordingly, the present disclosure provides a compound for use in a method of treating sickle cell disease in a subject in need thereof, wherein the compound is Compound I, or a pharmaceutically acceptable salt thereof, and wherein the method is as described herein.

Some embodiments of the present disclosure provide a compound for use in a method of treating sickle cell disease in a subject in need thereof, wherein the compound is Compound I of formula:

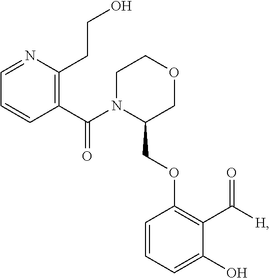

or a pharmaceutically acceptable salt thereof, and wherein the method comprises administering to the subject a first dose of Compound I, or a pharmaceutically acceptable salt thereof, for eight or more days, followed thereafter by a second dose of Compound I, or a pharmaceutically acceptable salt thereof, wherein the administrations of the first dose and the second dose provide about 25% to about 60% hemoglobin occupancy by Compound I.

In some embodiments of the compound for use, the administration of the first dose and the second dose of Compound I, or a pharmaceutically acceptable salt thereof, provide about 30% to about 50% hemoglobin occupancy by Compound I, or a pharmaceutically acceptable salt thereof.

Some embodiments of the present disclosure provide a compound for use in a method of treating sickle cell disease in a subject in need thereof wherein the compound is Compound I of formula:

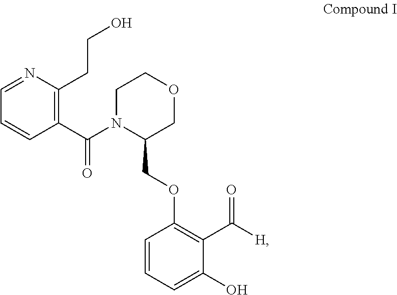

or a pharmaceutically acceptable salt thereof, and wherein the method comprises administering to the subject a first dose of Compound I, or a pharmaceutically acceptable salt thereof, for eight or more days, followed thereafter by a second dose of about 25 mg per day to about 500 mg per day of Compound I, or a pharmaceutically acceptable salt thereof.

Some embodiments of the present disclosure provide a compound for use in a method of treating sickle cell disease in a subject in need thereof, wherein the compound is Compound I of formula:

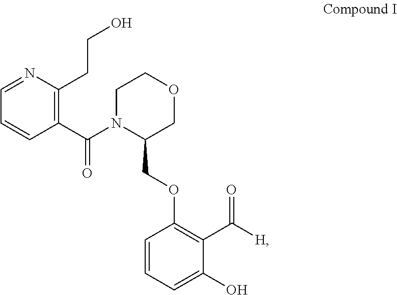

or a pharmaceutically acceptable salt thereof, and wherein the method comprises administering to the subject a first dose of about 300 mg once daily (QD) of Compound I, or a pharmaceutically acceptable salt thereof, for fourteen days, followed thereafter by a second dose of 200 mg per day of Compound I, or a pharmaceutically acceptable salt thereof.

In some embodiments of the methods described herein, or some embodiments of the compound for use in a method of treating sickle cell disease, the first dose of Compound I, or a pharmaceutically acceptable salt thereof, is administered once daily (QD), twice daily (BID), three times daily (TID), four times daily (QID), or every six hours (Q4H). In some embodiments of the methods described herein, or some embodiments of the compound for use, the first dose of Compound I, or a pharmaceutically acceptable salt thereof, is administered once daily (QD). In some embodiments of the methods described herein, or some embodiments of the compound for use, the first dose of Compound I, or a pharmaceutically acceptable salt thereof, is administered twice daily (BID). In some embodiments of the methods described herein, or some embodiments of the compound for use, the first dose of Compound I, or a pharmaceutically acceptable salt thereof, is administered three times daily (TID). In some embodiments of the methods described herein, or some embodiments of the compound for use, the first dose of Compound I, or a pharmaceutically acceptable salt thereof, is administered four times daily (QID). In some embodiments of the methods described herein, or some embodiments of the compound for use, the first dose of Compound I, or a pharmaceutically acceptable salt thereof, is administered every six hours (Q4H).

In some embodiments of the compound for use in a method of treating sickle cell disease, the first dose of Compound I, or a pharmaceutically acceptable salt thereof, is administered for fourteen days.

In some embodiments of the compound for use in a method of treating sickle cell disease, the second dose of Compound I, or a pharmaceutically acceptable salt thereof, is administered for at least one week, at least ten weeks, or at least fifty weeks. In some embodiments of the compound for use, the second dose of Compound I, or a pharmaceutically acceptable salt thereof, is administered for at least one week. In some embodiments of the compound for use, the second dose of Compound I, or a pharmaceutically acceptable salt thereof, is administered for at least ten weeks. In some embodiments of the compound for use, the second dose of Compound I, or a pharmaceutically acceptable salt thereof, is administered for at least fifty weeks.

In some embodiments of the compound for use in a method of treating sickle cell disease, the second dose of Compound I, or a pharmaceutically acceptable salt thereof, is administered for at least one year, at least five years, or at least ten years. In some embodiments of the compound for use, the second dose of Compound I, or a pharmaceutically acceptable salt thereof, is administered for at least one year.

In some embodiments of the compound for use, the second dose of Compound I, or a pharmaceutically acceptable salt thereof, is administered for at least five years. In some embodiments of the compound for use, the second dose of Compound I, or a pharmaceutically acceptable salt thereof, is administered for at least ten years.

In some embodiments of the compound for use in a method of treating sickle cell disease, the second dose of Compound I, or a pharmaceutically acceptable salt thereof, is administered for the lifetime of the subject. In some embodiments of the compound for use, the second dose of Compound I, or a pharmaceutically acceptable salt thereof, is administered until the subject dies.

In some embodiments of the compound for use, the second dose of Compound I, or a pharmaceutically acceptable salt thereof, is administered once daily (QD).

In some embodiments of the compound for use in a method of treating sickle cell disease, the percent hemoglobin occupancy in the subject, as provided by the administration of Compound I, or a pharmaceutically acceptable salt thereof, reaches about 25% to about 60% after about 8 hours to about 24 hours following the completion of the first dose (or loading dose) of Compound I, or a pharmaceutically acceptable salt thereof. In some embodiments of the compound for use, the percent hemoglobin occupancy in the subject, as provided by the administration of Compound I, or a pharmaceutically acceptable salt thereof, reaches about 25% to about 60% after 8 hours to about 20 hours following the completion of the first dose (or loading dose) of Compound I, or a pharmaceutically acceptable salt thereof. In some embodiments of the compound for use, the percent hemoglobin occupancy in the subject, as provided by the administration of Compound I, or a pharmaceutically acceptable salt thereof, reaches about 25% to about 60% after 8 hours to about 16 hours following the completion of the first dose (or loading dose) of Compound I, or a pharmaceutically acceptable salt thereof. In some embodiments of the compound for use, the percent hemoglobin occupancy in the subject, as provided by the administration of Compound I, or a pharmaceutically acceptable salt thereof, reaches about 25% to about 60% after 8 hours to about 12 hours following the completion of the first dose (or loading dose) of Compound I, or a pharmaceutically acceptable salt thereof. In some embodiments of the compound for use, the administration of the first dose and the second dose of Compound I provide about 30% to about 50% hemoglobin occupancy by Compound I

In some embodiments of the compound for use in a method of treating sickle cell disease, the percent hemoglobin occupancy in the subject, as provided by the administration of Compound I, or a pharmaceutically acceptable salt thereof, reaches about 25% to about 60% following a loading dose regimen as described herein. For example, in some embodiments of the compound for use, the percent hemoglobin occupancy in the subject, as provided by administration of Compound I, or a pharmaceutically acceptable salt thereof, reaches about 25% to about 60% following a loading dose regimen that comprises administration of 300 mg of Compound I, or a pharmaceutically acceptable salt thereof, once daily (QD) for fourteen days.

In some embodiments of the compound for use in a method of treating sickle cell disease, the percent hemoglobin occupancy in the subject, as provided by the administration of Compound I, or a pharmaceutically acceptable salt thereof, reaches about 25% to about 60% prior to administration of the second dose (or maintenance dose) of Compound I, or a pharmaceutically acceptable salt thereof. For example, in some embodiments of the compound for use, the percent hemoglobin occupancy in the subject, as provided by the administration of Compound I, or a pharmaceutically acceptable salt thereof, reaches about 25% to about 60% following a loading dose regimen that comprises administration of 300 mg of Compound I, or a pharmaceutically acceptable salt thereof, once daily (QD) for fourteen days, and prior to administration of the second dose (or maintenance dose) of Compound I, or a pharmaceutically acceptable salt thereof.

In some embodiments of the compound for use in a method of treating sickle cell disease, the administration of Compound I, or a pharmaceutically acceptable salt thereof, provides a hematocrit value of about 20% to about 50% in the subject.

In some embodiments of the compound for use in a method of treating sickle cell disease, the administration of Compound I, or a pharmaceutically acceptable salt thereof, provides a hematocrit value of at least about 20% in the subject. In some embodiment, the administration of Compound I, or a pharmaceutically acceptable salt thereof, provides a hematocrit value of at least about 30% in the subject. In some embodiment of the compound for use, the administration of Compound I, or a pharmaceutically acceptable salt thereof, provides a hematocrit value of about 40% in the subject. In some embodiment, the administration of Compound I, or a pharmaceutically acceptable salt thereof, provides a hematocrit value of about 45% in the subject.

In some embodiments of the compound for use in a method of treating sickle cell disease, the administration of Compound I, or a pharmaceutically acceptable salt thereof, provides a blood concentration of Compound I of about 50 μg/mL to about 800 μg/mL. In some embodiments of the compound for use in a method of treating sickle cell disease, the administration of Compound I, or a pharmaceutically acceptable salt thereof, provides a blood concentration of Compound I of about 95 μg/mL to about 775 μg/mL. In some embodiments of the compound for use in a method of treating sickle cell disease, the administration of Compound I, or a pharmaceutically acceptable salt thereof, provides a blood concentration of Compound I of about 95 μg/mL to about 750 μg/mL. In some embodiments of the compound for use in a method of treating sickle cell disease, the administration of Compound I, or a pharmaceutically acceptable salt thereof, provides a blood concentration of Compound I of about 140 μg/mL to about 700 μg/mL. In some embodiments of the compound for use in a method of treating sickle cell disease, the administration of Compound I, or a pharmaceutically acceptable salt thereof, provides a blood concentration of Compound I of about 190 μg/mL to about 650 μg/mL. In some embodiments of the compound for use in a method of treating sickle cell disease, the administration of Compound I, or a pharmaceutically acceptable salt thereof, provides a blood concentration of Compound I of about 250 μg/mL to about 475 μg/mL. In some embodiments of the compound for use in a method of treating sickle cell disease, the administration of Compound I, or a pharmaceutically acceptable salt thereof, provides a blood concentration of Compound I of about 300 μg/mL to about 450 μg/mL. In some embodiments of the compound for use in a method of treating sickle cell disease, the administration of Compound I, or a pharmaceutically acceptable salt thereof, provides a blood concentration of Compound I of about 300 μg/mL to about 475 μg/mL.

In some embodiments of the compound for use in a method of treating sickle cell disease, the administration of Compound I, or a pharmaceutically acceptable salt thereof, provides an AUC0-24 of Compound I of about 1,200 μg*h/mL to about 19,200 μg*h/mL. In some embodiments of the compound for use in a method of treating sickle cell disease, the administration of Compound I, or a pharmaceutically acceptable salt thereof, provides an AUC0-24 of Compound I of about 2,280 μg*h/mL to about 18,600 μg*h/mL. In some embodiments of the compound for use in a method of treating sickle cell disease, the administration of Compound I, or a pharmaceutically acceptable salt thereof, provides an AUC0-24 of Compound I of about 2,280 μg*h/mL to about 18,000 μg*h/mL. In some embodiments of the compound for use in a method of treating sickle cell disease, the administration of Compound I, or a pharmaceutically acceptable salt thereof, provides an AUC0-24 of Compound I of about 3,360 μg*h/mL to about 16,800 μg*h/mL. In some embodiments of the compound for use in a method of treating sickle cell disease, the administration of Compound I, or a pharmaceutically acceptable salt thereof, provides an AUC0-24 of Compound I of about 4,560 μg*h/mL to about 15,600 μg*h/mL. In some embodiments of the compound for use in a method of treating sickle cell disease, the administration of Compound I, or a pharmaceutically acceptable salt thereof, provides an AUC0-24 of Compound I of about 6,000 μg*h/mL to about 11,400 μg*h/mL. In some embodiments of the compound for use in a method of treating sickle cell disease, the administration of Compound I, or a pharmaceutically acceptable salt thereof, provides an AUC0-24 of Compound I of about 7,200 μg*h/mL to about 10,800 μg*h/mL. In some embodiments of the compound for use in a method of treating sickle cell disease, the administration of Compound I, or a pharmaceutically acceptable salt thereof, provides an AUC0-24 of Compound I of about 7,200 μg*h/mL to about 11,400 μg*h/mL.

In some embodiments of the compound for use in a method of treating sickle cell disease, the administration of Compound I, or a pharmaceutically acceptable salt thereof, provides an AUC0-24 of Compound I of more than about 1,200 μg*h/mL. In some embodiments of the compound for use in a method of treating sickle cell disease, the administration of Compound I, or a pharmaceutically acceptable salt thereof, provides an AUC0-24 of Compound I of more than about 2,280 μg*h/mL. In some embodiments of the compound for use in a method of treating sickle cell disease, the administration of Compound I, or a pharmaceutically acceptable salt thereof, provides an AUC0-24 of Compound I of more than about 3,360 μg*h/mL. In some embodiments of the compound for use in a method of treating sickle cell disease, the administration of Compound I, or a pharmaceutically acceptable salt thereof, provides an AUC0-24 of Compound I of more than about 4,560 μg*h/mL. In some embodiments of the compound for use in a method of treating sickle cell disease, the administration of Compound I, or a pharmaceutically acceptable salt thereof, provides an AUC0-24 of Compound I of more than about 6,000 μg*h/mL. In some embodiments of the compound for use in a method of treating sickle cell disease, the administration of Compound I, or a pharmaceutically acceptable salt thereof, provides an AUC0-24 of Compound I of more than about 7,200 μg*h/mL. In some embodiments of the compound for use in a method of treating sickle cell disease, the administration of Compound I, or a pharmaceutically acceptable salt thereof, provides an AUC0-24 of Compound I of more than about 10,800 μg*h/mL. In some embodiments of the compound for use in a method of treating sickle cell disease, the administration of Compound I, or a pharmaceutically acceptable salt thereof, provides an AUC0-24 of Compound I of more than about 11,400 μg*h/mL. In some embodiments of the compound for use in a method of treating sickle cell disease, the administration of Compound I, or a pharmaceutically acceptable salt thereof, provides an AUC0-24 of Compound I of more than about 15,600 μg*h/mL. In some embodiments of the compound for use in a method of treating sickle cell disease, the administration of Compound I, or a pharmaceutically acceptable salt thereof, provides an AUC0-24 of Compound I of more than about 16,800 μg*h/mL. In some embodiments of the compound for use in a method of treating sickle cell disease, the administration of Compound I, or a pharmaceutically acceptable salt thereof, provides an AUC0-24 of Compound I of more than about 18,000 μg*h/mL. In some embodiments of the compound for use in a method of treating sickle cell disease, the administration of Compound I, or a pharmaceutically acceptable salt thereof, provides an AUC0-24 of Compound I of more than about 18,600 μg*h/mL. In some embodiments of the compound for use in a method of treating sickle cell disease, the administration of Compound I, or a pharmaceutically acceptable salt thereof, provides an AUC0-24 of Compound I of more than about 19,000 μg*h/mL.

In some embodiments of the compound for use in a method of treating sickle cell disease, the first dose of Compound I, or a pharmaceutically acceptable salt thereof, is administered as a pharmaceutical composition. In some embodiments of the compound for use in a method of treating sickle cell disease, the second dose of Compound I, or a pharmaceutically acceptable salt thereof, is administered as a pharmaceutical composition.

In some embodiments of the compound for use in a method of treating sickle cell disease, Compound I, or a pharmaceutically acceptable salt thereof, is administered orally.

In some embodiments of the compound for use in a method of treating sickle cell disease, Compound I is a crystalline form characterized by an X-ray powder diffractogram comprising the following peaks: 18.3°, 23.4°, and 26.1° 2θ±0.2° 2θ (Compound I Form I), as determined on a diffractometer using Cu-Kα radiation.

In some embodiments of the compound for use in a method of treating sickle cell disease, the diffractogram further comprises one or more peaks at: 10.8° or 17.3° 2θ±0.2° 2θ.

In some embodiments of the compound for use in a method of treating sickle cell disease, the crystalline form is characterized by a differential scanning calorimetry (DSC) curve comprising an endotherm at about 111° C. (onset temperature).

In some embodiments of the compound for use in a method of treating sickle cell disease, the use in treating sickle cell disease further comprises administering an additional therapeutic agent.

In some embodiments of the compound for use in a method of treating sickle cell disease, the additional therapeutic agent is a modulator of hemoglobin. In some embodiments, the additional therapeutic agent is useful for treating sickle cell disease. In some embodiments, the additional therapeutic agent is useful for treating a complication of sickle cell disease. Non-limiting examples of a complication of sickle cell disease include iron overload, pain, infections, acute chest syndrome, stroke, and pulmonary hypertension. In some embodiments, the additional therapeutic agent is hydroxyurea, L-glutamine, crizanlizumab, or deferiprone. In some embodiments, the additional therapeutic agent is hydroxyurea.

In some embodiments of the compound for use in a method of treating sickle cell disease, the additional therapeutic agent is hydroxyurea.

### 3. Pharmaceutical Compositions and Modes of Administration

Compound I, or a pharmaceutically acceptable salt thereof, or a crystalline form thereof, as described herein may be administered in a pharmaceutical composition. Thus, provided herein are pharmaceutical compositions comprising one or more of the forms of Compound I described herein, or salts or solvates thereof, and one or more pharmaceutically acceptable vehicles such as carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modem Pharmaceutics, Marcel Dekker, Inc. 3rd Ed. (G. S. Banker & C. T. Rhodes, Eds.).

The pharmaceutical compositions may be administered alone or in combination with other therapeutic agents.

The pharmaceutical compositions may be administered in either single or multiple first and/or second doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein, solid forms described herein, or salts or solvates thereof. Administration may be via, for example, capsule or enteric coated tablets. In making the pharmaceutical compositions that include at least one compound described herein, solid form described herein, or salt or solvate thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active ingredient, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound or solid form described herein, or salts or solvates thereof, can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

Another formulation for use in the methods disclosed herein employ transdermal delivery devices (“patches”). Such transdermal patches may be used to provide continuous or discontinuous infusion of the compounds or solid forms described herein, or salts or solvates thereof, in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on-demand delivery of pharmaceutical agents.

For preparing solid compositions such as tablets, the principal active ingredient may be mixed with a pharmaceutical excipient to form a solid preformulation composition containing a homogeneous mixture of a compound or solid form described herein, or salts or solvates thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds or solid forms described herein, or salts or solvates thereof, may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, acetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device, or the nebulizing device may be attached to a facemask tent, or intermittent positive pressure breathing machine. Solution, suspension, or powder compositions may be administered, preferably orally or nasally, from devices that deliver the formulation in an appropriate manner.

### EXAMPLES

### Example 1: A Phase 2/3 Randomized and Multicenter Study of Compound I Administered Orally to Participants with Sickle Cell Disease and an Open-Label Pharmacokinetics Study in Pediatric Participants with Sickle Cell Disease

This example describes a three-part Phase 2/3 study of orally administered Compound I to participants with SCD.

### Part A

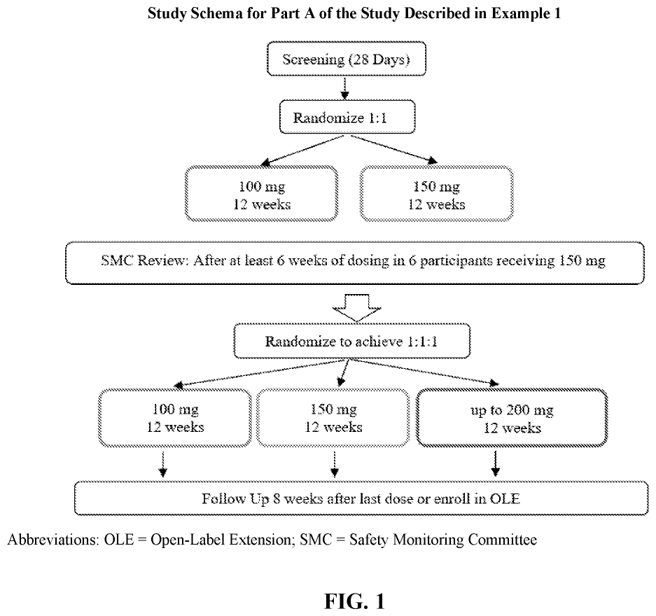

Part A of this study is a Phase 2 randomized, open-label, dose-finding study in approximately 60 adult participants with SCD. The primary objective of Part A of this study is to assess the effects of Compound I in adult participants with SCD as measured by change in hemoglobin. A study schema for Part A of the study is shown in FIG. 1.

Participants are screened for eligibility in the study from Day −28 through Day −1. Eligible participants are randomized on Day 1 based on a 1:1 (100 mg: 150 mg) allocation and will receive their initial dose (loading dose) of Compound I. After completion of Day 1 assessments and subsequent loading doses, participants will continue with administration of their maintenance dose daily through Week 12.

After completion of at least 6 weeks of dosing of 6 adult participants from the 150 mg treatment group, safety, tolerability, and available PK, Hb occupancy, and PD data is reviewed to assess whether the maintenance dose may be increased up to a maximum of 200 mg.

After a determination has been made to include a higher dose treatment arm, participants enrolled thereafter are randomized 1:1:1 (100 mg: 150 mg:up to 200 mg of Compound I respectively) or adaptive method to ensure balance and receive a loading dose regimen over 4 days followed by daily maintenance doses through Week 12. Loading doses to be used in this study will not exceed 2200 mg within 24 hours period.

The proposed loading and maintenance doses to achieve the desired exposures and targeted % Hb occupancies for each dose group are shown in Table 1 below.

TABLE 1
Model-Predicted Dosing Regimen

| Dose | Sample | Loading | Maintenance | Mean Targeted % |
|---|---|---|---|---|
| Group | Size (N) | Doses | Dose | HbOccupancy^{a} |
| 1 | 20 | 200 mg BID × | 100 mg QD | ~32% |
| | | 4 days | | |
| 2 | 20 | 300 mg BID × | 150 mg QD | ~46% |
| | | 4 days | | |
| 3 | 10 | 300 mg QD × | 200 mg QD | 59%^{b} |
| | | 14 days | | |

^{a}Mean targeted % Hb occupancy values incorporate changes in hemoglobin (ΔHb) and hematocrit (ΔHct) based on observed changes in Hb and Hct in SCD patients treated with Compound I.
^{b}Based on a model that assumes an equivalent change from baseline in Hb and Hct as observed in the 150 mg maintenance dose group

### Part A Sub Study 1

Selective participants in Part A will non-invasively have their endogenous carbon monoxide levels measured through a breath test in order to calculate RBC lifespan in response to Compound I treatment.

Part A Sub study 2

Some newly enrolled participants in Part A will receive a loading dose regimen of 300 mg of Compound I once daily over the first 14 days. Participants will then receive 200 mg once daily maintenance doses through Week 12.

### Endpoints

The primary endpoint of Part A of the study is to assess the change from baseline in hemoglobin through Week 12.

The secondary endpoints of Part A of the study include the effects of Compound I on hemoglobin, hematocrit, clinical measures of hemolysis, and the safety and tolerability, as well as the PK and PD properties of multiple dose Compound I administration. Additional secondary endpoints of Part B of the study also include:
Proportion of participants with an increase from baseline of >1 g/dL in Hb through Week 12
Percent change from baseline in hemolysis measures, including indirect bilirubin, absolute and % reticulocytes, and LDH through Week 12
Incidence of adverse events, changes in laboratory assessments, ECGs, and vital signs
Effect on Hb OEC as measured by p50 through Week 12, and
AUC₀₋₂₄, Cₘₐₓ, Tₘₐₓ, blood to plasma (B:P) ratios of these PK parameters (except Tₘₐₓ) after the first dose.

The exploratory endpoint of Part A of the study is to evaluate the effects of Compound I on neurocognitive function, erythropoietin (EPO) levels, quality of life (QOL) assessments, RBC deformability, RBC mitochondrial content, VOCs, and other biomarkers. Additional exploratory endpoints of Part A of the study include:
Change from baseline through Week 12 in the Executive abilities composite score (using Dimensional Change Card Sort Test, Flanker Inhibitory Control and Attention Test, and Pattern Comparison Processing Speed Test as assessed by the NIH Toolbox Cognition Module).
Change from baseline in EPO levels through Week 12.
Change from baseline in QOL assessments through Week 12 including PGI-C, CGI-C, EQ-5D-5L, and PROMIS-29.
Change from baseline in RBC deformability and RBC mitochondrial content through Week 12.
Annualized rate of VOC through Week 12.
Time to first VOC from randomization.
Change from baseline in other biomarkers at Week 12 (including genomic analysis, membrane fragility, flow adhesion, and protein markers by multiplex assay which may include ICAMs, VCAMs, P-selectin, E-selectin, ACR, KIM, and markers of hemolysis such as levels of haptoglobin, hemopexin, soluble C3, and cell free hemoglobin).

### Part B

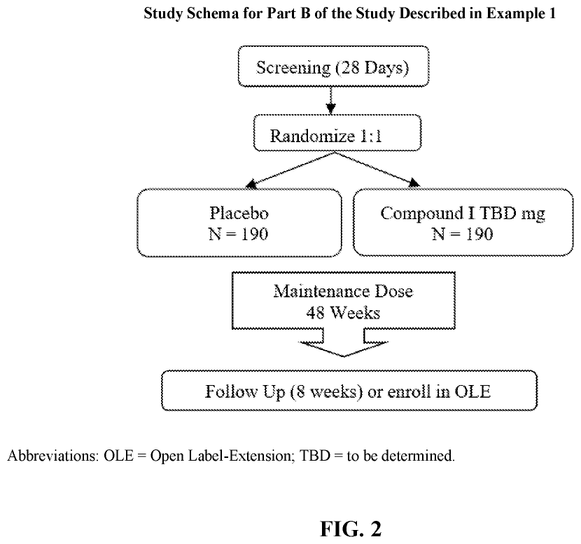

Part B is a Phase 3, randomized, double-blind, placebo-controlled study in adult and pediatric participants. Part B will initiate after selection of the optimum dose from Part A of the study. Part B of the study will assess the safety and efficacy of Compound I in increasing the Hb response in approximately 380 adult and pediatric participants with SCD, with approximately 190 participants receiving placebo and approximately 190 participants receiving Compound I optimal dose selected from Part A of the study. A study schema for Part B of the study is shown in FIG. 2.

The primary objective of Part B of the study is to assess the effects of an optimum Compound I dose as compared to placebo in adult and pediatric participants with SCD as measured by hemoglobin response.

In Part B of the study, participants are screened for eligibility in the study from Day −28 through Day −1. Eligible adult participants are randomized on Day 1 based on a 1:1 (Compound I:Placebo) allocation and will receive their initial dose (loading dose) at the clinical site. After completion of Day 1 assessments and subsequent loading doses, participants will continue with administration of their maintenance dose daily from Week 1 through Week 48. Participants will return to the clinical site for assessments of safety, efficacy, and PK/PD. The incidence of VOC events will be collected every 4 weeks. After completion of treatment at Week 48 (Day 337), participants will return for final follow-up visit 8 weeks later (Week 56, Day 393), approximately 5 half-lives after the last dose. Participants may choose to enroll in an OLE study under a separate protocol after completing Week 48 visits. Participants not entering the OLE study will be followed for approximately 8 weeks after completing dosing to further assess safety.

### Part B Sub Study

Selective participants in Part B will non-invasively have their endogenous carbon monoxide levels measured through a breath test in order to calculate RBC lifespan in response to Compound I treatment.

### Endpoints

The primary endpoint of Part B of the study is to assess the proportion of participants with an increase from baseline of >1 g/dL in Hb at Week 48.

The secondary endpoints of Part B of the study include the effects of Compound I compared to placebo on hemoglobin, hematocrit, clinical measures of hemolysis, and relevant clinical outcomes, and to evaluate the safety and tolerability of 48 weeks of daily administration of Compound I. Additional secondary endpoints also include:
Annualized rate of VOC through end of Week 48.
Proportion of participants with an increase from baseline of >1 g/dL in Hb at Week 24.
Change from baseline in Hb through Weeks 24 and 48.
Percent change from baseline in hemolysis measures, including indirect bilirubin, absolute and % reticulocytes, and LDH through Week 48.
Change from baseline in QOL assessments through Week 48 including PGI-C, CGI-C, and PROMIS-29 physical function, anxiety, depression, fatigue, social function, pain interference, and pain intensity.
Incidence of adverse events, changes in laboratory assessments, ECGs, and vital signs.
Trough plasma and blood concentrations, B:P ratio.
Percent Hb occupancy through Week 48.
Time to first VOC from randomization.
Time to first Hb increase from baseline of >1 g/dL during the study.

The exploratory endpoint of Part B of the study is to evaluate the effects of Compound I as compared to placebo on neurocognitive function, EPO levels, QOL assessments, RBC deformability, RBC mitochondrial content, VOCs, and renal biomarkers. Additional exploratory endpoints also include:
Change from baseline in other biomarkers through Week 48 (including genomic analysis [optional] membrane fragility, flow adhesion, and protein markers by multiplex assay which may include ICAMs, VCAMs, P-selectin, E-selectin, ACR, KIM, and markers of hemolysis such as levels of haptoglobin, hemopexin, soluble C3, and cell free hemoglobin).
Change from baseline through Week 48 in the Executive abilities composite score (using Dimensional Change Card Sort Test, Flanker Inhibitory Control and Attention Test, and Pattern Comparison Processing Speed Test) as assessed by the NIH Toolbox Cognition Module.
Change from baseline in EPO levels through Week 48.
Change from baseline in QOL assessments through Week 48 including EQ-5D-5L.
Change from baseline in RBC deformability and mitochondrial content through Week 48.

### Part C

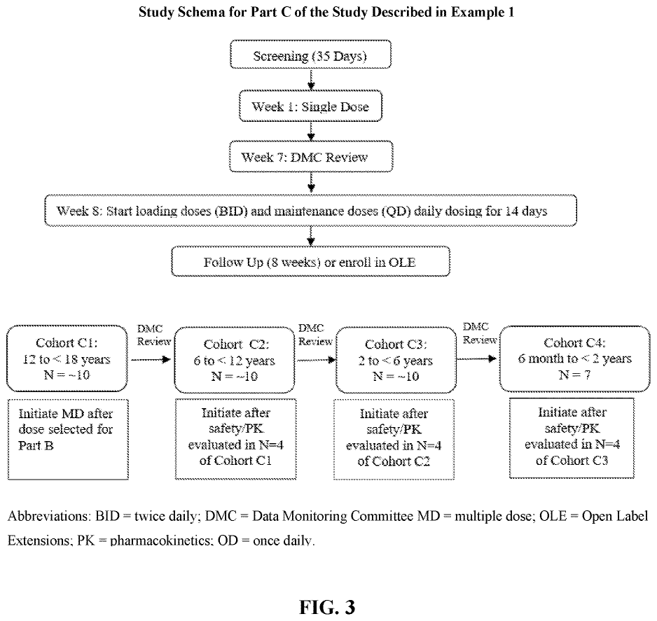

Part C of the study is a single arm, open-label, multi-dose PK study in four age-group cohorts of pediatric participants with SCD, and the multi-dose portion of Part C of the study will begin after the dose has been selected for Part B of the study. A study schema for Part B of the study is shown in FIG. 3.

Pediatric participants are assessed for study eligibility during a 35-day Screening Period (additional time allowed in Screening due to blood draw volume restrictions in pediatric participants). On study Day 1, eligible pediatric participants will receive a single dose of Compound I (100 mg dose in cohort C1, dose level for cohorts C2, C3, and C4 to be determined based on emerging data). Safety, tolerability, and available PK data of Compound I will be assessed approximately 7 weeks after at least 4 participants have completed single-dose (SD) administration in each Cohort and provide a recommendation on proceeding with multiple-dose (MD) dosing. Pediatric participants will receive a loading dose regimen over 4 days (starting on MD Day 1) followed by once daily dosing for a total treatment duration of 14 days. Doses given in the MD portion will be informed by emerging clinical trial data and will not exceed dose levels established as safe in adult clinical trials or previous pediatric cohorts. Participants may choose to enroll in an open-label extension (OLE) study under a separate protocol after completing MD Week 2 visits. Participants not entering the OLE study will be followed for approximately 8 weeks after completing dosing to further assess safety.

Part C is composed of four cohorts:
Cohort C1 includes approximately 10 participants aged 12 to less than 18 years, at least 30% of whom must be 12 to less than 15 years old.
Cohort C2 includes approximately 10 participants aged 6 to less than 12 years, at least 30% of whom must be 6 to less than 9 years old.
Cohort C3 includes approximately 10 participants aged 2 to less than 6 years.
Cohort C4 includes approximately 7 participants aged 6 months to less than 2 years.

Cohorts C2-C4 will be initiated after completion of 14 days of MD dosing and Data Monitoring Committee (DMC) review of safety, tolerability, and PK data has been evaluated in at least 4 participants of the preceding Cohort. Up to 10 additional participants may be enrolled in each cohort if needed to evaluate additional dose levels and/or to further characterize PK or safety if deemed necessary. Participants from Part C will not be eligible to enroll in Part B of the study.

### Inclusion Criteria

Key inclusion criteria for Parts A, B, and C include:
Male or female with SCD. Documentation of SCD genotype homozygous for sickle cell allele (HbSS) or double heterozygote for sickle hemoglobin (HbS) and β-0 thalassemia (HbSB) may be based on history of laboratory testing or must be confirmed by laboratory testing during Screening.
Hb≥5.5 and ≤10.5 g/dL during Screening and considered stable by the Investigator.
For participants taking hydroxyurea (HU) and/or L-glutamine, the dose must be stable for at least 90 days prior to signing the ICF or assent and with no anticipated need for dose adjustments during the study in the opinion of the Investigator.
Female participants of child-bearing potential, must agree to use highly effective methods of contraception or practice abstinence from study start to 120 days after the last dose of study drug. Males who are not surgically sterile with partners of childbearing potential must agree to use a highly effective method of birth control during the study and for 120 days after the last dose of study drug. In addition, males who are not surgically sterile with a partner who is pregnant must agree to condom use or maintain sexual abstinence during the study and for 120 days after the last dose of study drug.
Female participants of child-bearing potential must have a negative pregnancy test before administration of study drug.
Participant has provided documented informed consent (Part A and Part B for adult participants) or written informed parental/guardian consent and participant assent has been obtained per institutional review board (IRB)/Ethics Committee (EC) policy and requirements, consistent with International Council for Harmonisation (ICH) guidelines (Part B for pediatric participants and Part C).

Additional key inclusion criteria for Part A include:
Age 18 to ≤65 years, inclusive at Screening
Males must agree to not donate sperm from study start through 120 days after final dose.

Additional key inclusion criteria for Part B include:
Age 12 to ≤65 years, inclusive at Screening. Participants 12 to <18 years will only be enrolled in Part B after evaluation of safety in Part C Cohort C1.
Males must agree to not donate sperm from study start through 120 days after the final dose
More than or equal to 2 and ≤10 VOCs within 12 months of Screening.

### Exclusion Criteria

The key exclusion criteria for Parts A, B, and C include:
More than 10 VOCs within 12 months of Screening.
Female participant who is breastfeeding or pregnant.
Receiving regularly scheduled RBC transfusion therapy (also termed chronic, prophylactic, or preventive transfusion) or has received an RBC or exchange transfusion for any reason within 90 days of Day 1.
Hospitalized for sickle cell crisis or other vaso-occlusive event within 14 days of signing the ICF.
Screening laboratory test of alanine aminotransferase (ALT)>4× upper limit of normal (ULN) for age.
Acute illness or clinically significant bacterial, fungal, parasitic, or viral infection which requires therapy, including acute bacterial infection requiring antibiotics within 14 days prior to the study drug administration.
Participants known to have active hepatitis A, B, or C or human immunodeficiency virus (HIV).
Participants with active, symptomatic coronavirus disease 2019 (COVID-19) infection.
Estimated glomerular filtration rate (eGFR)<60 mL/min/1.73 m² at the Screening Visit, calculated by the central laboratory, or is on chronic dialysis.
History of malignancy within the past 2 years prior to treatment Day 1 requiring chemotherapy and/or radiation (with the exception of local therapy for non-melanoma skin malignancy).
History of unstable or deteriorating cardiac or pulmonary disease within 6 months prior to consent.
Has received COVID-19 vaccine (first dose, second dose, or booster dose), authorized by regional regulatory authority, within 7 days prior to Day 1
Received EPO or other hematopoietic growth factor treatment within 28 days of signing ICF or is anticipated to require such agents during the study.
Current or recent use of voxelotor. Recent use is defined as within 10 days prior to Day 1.
Current or recent use of crizanlizumab. Recent use is defined as within 90 days prior to Day 1.
Ongoing or recent use of strong or moderate inducers of cytochrome P450 (CYP) or CYP3A4/CYP3A5. Recent is defined as within 5 elimination half-lives or 14 days, whichever is longer prior to Day 1.
Ongoing or recent use of strong or moderate inhibitors of CYP3A4/CYP3A5. Recent is defined as within 5 elimination half-lives prior to Day 1.
Ongoing or recent use of the P-glycoprotein substrates digoxin or dabigatran. Recent is defined as within 5 elimination half-lives prior to Day 1.
Use of prohibited prescription or nonprescription drugs and dietary supplements (including herbal and alternative medications). Marijuana use is allowed, except for 24 hours prior to neurocognitive assessments as outlined in the Schedule of Assessments.
Known allergy to Compound I or other Hb polymerization inhibitors.
History of severe allergic reaction (including anaphylaxis) to any substance, or previous status asthmaticus.
Unlikely to comply with the study procedures

Additional key exclusion criteria for Part C only include:
History of stroke or meeting criteria for primary stroke prophylaxis (history of two transcranial doppler [TCD] measurements ≥200 cm/sec by nonimaging TCD or ≥185 cm/sec by TCDi).

### Dose

Dose selection is based on safety and PK results from previous studies as described in Examples 1, 2, and 3.

A loading dose is administered followed by daily maintenance dosing for multi-dose regimens.

### Duration of Treatment

In Part A, all adult participants will receive maintenance dosing through approximately Week 12.

In Part B, all adult and pediatric participants will receive maintenance dosing for 48 weeks.

In Part C, pediatric participants in Cohort C1 will receive a single dose of 100 mg followed by daily dosing starting at approximately Week 8 continuing for 14 days. The multi-day dosing regimen for all Part C cohorts and single dose levels for Cohorts C2 to C4 will be informed by emerging data. Doses utilized will not exceed doses determined to have acceptable tolerability in adults or older age pediatric cohorts. Part C cohorts C3 and C4 will be administered Compound I as either a powder for oral solution, or a dispersible tablet.

### Preliminary Results

Table 2 below includes the preliminary blood analysis of Patient A who was administered Compound I according to the dosing regimen described above in Part A Sub Study 2. Patient A is male and 61 years old.

TABLE 2
Preliminary Blood Analysis of Patient A

| | Reference | | | | | | | | |
|---|---|---|---|---|---|---|---|---|---|
| | Range | Day 0 | Day | Day | Day | Day | Day | Day | Day |
| Test Type | (units) | (screening) | 1 | 8 | 15 | 29 | 43 | 57 | 85 |
| Hemoglobin | 13.5-17.5 | 7.0 | 8.8 | N/A | 10.6 | 12.2 | 14.9 | 12.4 | 11.0 |
| (HGB) | (g/dL) | | | | | | | | |
| Hematocrit | 38-53 | 20.4 | 25.1 | N/A | 30.9 | 35.9 | 43.3 | 36.3 | 31.0 |
| (HCT) | (%) | | | | | | | | |
| Platelet count | 150-450 | 148 | 219 | N/A | 239 | 211 | 249 | 153 | 146 |
| | (K/μL) | | | | | | | | |
| Reticulocytes | 0.5-1.5 | 2.2 | 5.2 | N/A | 3.0 | 2.0 | 1.5 | 0.1 | 5.2 |
| | (%) | | | | | | | | |
| Absolute | 1.5-7.7 | 1.7 | 2.2 | N/A | 2.2 | 0.5 | 0.6 | 3.5 | 1.1 |
| neutrophil | (K/μL) | | | | | | | | |
| Bilirubin total | 0-1.3 | 2.4 | 2.16 | N/A | 1.06 | N/A | 1.06 | 2.77 | 2.89 |
| | (mg/dL) | | | | | | | | |
| Bilirubin direct | 0-0.2 | 0.48 | <0.01 | N/A | <0.01 | N/A | 0.28 | 0.38 | 0.08 |
| | (mg/dL) | | | | | | | | |
| Bilirubin indirect | (mg/dL) | 1.92 | N/A | N/A | N/A | N/A | 0.78 | 2.39 | 2.81 |
| Alkaline | 35.0-128.0 | 143.5 | 152.7 | N/A | 156.7 | N/A | 160.8 | 198.2 | 150.6 |
| phosphatase | (IU/L | | | | | | | | |
| Aspartate | 12-60 | 56.0 | 60.0 | N/A | 43.1 | N/A | 44.1 | 82.9 | 54.0 |
| Aminotransferase | (IU/L) | | | | | | | | |
| (AST) | | | | | | | | | |
| Alanine | 10-49 | 26.1 | 28.2 | N/A | 26.1 | N/A | 28.9 | 66.6 | 28.2 |
| Aminotransferase | (IU/L) | | | | | | | | |
| (ALT) | | | | | | | | | |
| Lactate | 111-231 | 358 | 339 | N/A | 243 | N/A | 193 | 345 | 354 |
| dehydrogenase | (IU/L) | | | | | | | | |
| (LDH) | | | | | | | | | |

Based on preliminary pharmacokinetic (PK) analysis of whole blood samples taken from Patient A at day 15 and day 29, Hb occupancy values of 62% and 53%, respectively, were calculated.

Table 3 below includes preliminary blood analysis of Patient B who was administered Compound I according to the dosing regimen described above in Part A Sub Study 2. Patient B is female and 20 years old.

TABLE 3
Preliminary Blood Analysis of Patient B

| | Reference | | | | | | | | |
|---|---|---|---|---|---|---|---|---|---|
| | Range | Day 0 | Day | Day | Day | Day | Day | Day | Day |
| Test Type | (units) | (screening) | 1 | 8 | 15 | 29 | 43 | 57 | 85 |
| Hemoglobin | 12-15.5 | 8.3 | 8.5 | 11.2 | N/A | 14.4 | 15.2 | 14.3 | 14.9 |
| (HGB) | (g/dL) | | | | | | | | |
| Hematocrit (HCT) | 36-46 | 23.5 | 24.7 | 33.1 | N/A | 42.8 | 45.4 | 42.6 | 43.7 |
| | (%) | | | | | | | | |
| Platelet count | 150-450 | 360 | 344 | 411 | N/A | 333 | 260 | 290 | 256 |
| | (K/μL) | | | | | | | | |
| Reticulocytes | 0.5-1.5 | 11.5 | 9.5 | 5.4 | N/A | 1.2 | 0.8 | 0.9 | 1.1 |
| | (%) | | | | | | | | |
| Absolute neutrophil | 1.5-7.7 | 6.9 | 7.3 | 4.1 | N/A | 4.1 | 3.7 | 5.5 | 5.6 |
| | (K/μL) | | | | | | | | |
| Bilirubin total | 0-1.3 | 4.84 | 4.35 | 2.4 | N/A | 1.06 | 0.69 | 0.57 | 0.94 |
| | (mg/dL) | | | | | | | | |
| Bilirubin direct | 0-0.2 | 0.18 | 0.18 | 0.08 | N/A | 0.18 | 0.18 | 0.18 | <0.01 |
| | (mg/dL) | | | | | | | | |
| Bilirubin indirect | (mg/dL) | 4.66 | 4.17 | 2.32 | N/A | 0.88 | 0.51 | 0.39 | N/A |
| Alkaline | 33.0-111.0 | 49.3 | 52.4 | 58.5 | N/A | 67.6 | 67.6 | 69.6 | 72.6 |
| phosphatase | (IU/L) | | | | | | | | |
| Aspartate | 12-50 | 41.1 | 39.1 | 42.1 | N/A | 29.1 | 27.1 | 26.2 | 27.1 |
| Aminotransferase | (IU/L) | | | | | | | | |
| (AST) | | | | | | | | | |
| Alanine | 10-39 | 22.0 | 22.0 | 21.3 | N/A | 17.2 | 16.5 | 17.9 | 20.0 |
| Aminotransferase | (IU/L) | | | | | | | | |
| (ALT) | | | | | | | | | |
| Lactate | 111-231 | 371 | 351 | 385 | N/A | 195 | 151 | 152 | 141 |
| dehydrogenase | (IU/L) | | | | | | | | |
| (LDH) | | | | | | | | | |

Table 4 below includes preliminary blood analysis of Patient C who was administered Compound I according to the dosing regimen described above in Part A Sub Study 2. Patient C is female and 27 years old.

TABLE 4
Preliminary Blood Analysis of Patient C

| | Reference | | | | | | | | |
|---|---|---|---|---|---|---|---|---|---|
| | Range | Day 0 | Day | Day | Day | Day | Day | Day | Day |
| Test Type | (units) | (screening) | 1 | 8 | 15 | 29 | 43 | 57 | 85 |
| Hemoglobin | 12-15.5 | 7.1 | N/A | 9.1 | 10.2 | 11.6 | 10.5 | 10.4 | 10.0 |
| (HGB) | (g/dL) | | | | | | | | |
| Hematocrit | 36-46 | 21.0 | N/A | 25.9 | 29.8 | 33.9 | 30.1 | 28.9 | 28.2 |
| (HCT) | (%) | | | | | | | | |
| Platelet count | 150-450 | 392 | N/A | 405 | 482 | 357 | 416 | 413 | 457 |
| | (K/μL) | | | | | | | | |
| Reticulocytes | 0.5-1.5 | 13.7 | N/A | 9.9 | 5.0 | 1.8 | 2.8 | 4.2 | 5.9 |
| | (%) | | | | | | | | |
| Absolute | 1.5-7.7 | 0.8 | N/A | 6.7 | 3.9 | 2.6 | 1.0 | 4.3 | 4.9 |
| neutrophil | (K/μL) | | | | | | | | |
| Bilirubin total | 0-1.3 | 4.84 | 5.45 | 4.23 | 3.50 | 2.89 | 4.72 | 3.62 | 3.62 |
| | (mg/dL) | | | | | | | | |
| Bilirubin direct | 0-0.2 | 0.58 | 0.58 | 0.38 | 0.38 | 0.38 | 0.28 | 0.18 | <0.01 |
| | (mg/dL) | | | | | | | | |
| Bilirubin indirect | (mg/dL) | 4.26 | 4.87 | 3.85 | 3.12 | 2.51 | 4.44 | 3.44 | N/A |
| Alkaline | 33.0-111.0 | 52.4 | 51.4 | 61.5 | 54.4 | 51.4 | 58.5 | 56.4 | 55.4 |
| phosphatase | (IU/L) | | | | | | | | |
| Aspartate | 12-50 | 48.0 | 62.0 | 57.0 | 34.1 | 39.1 | 46.1 | 46.1 | 39.1 |
| Aminotransferase | (IU/L) | | | | | | | | |
| (AST) | | | | | | | | | |
| Alanine | 10-39 | 20.0 | 24.8 | 28.9 | 18.6 | 17.2 | 26.8 | 21.3 | 20.6 |
| Aminotransferase | (IU/L) | | | | | | | | |
| (ALT) | | | | | | | | | |
| Lactate | 111-231 | 533 | 691 | 545 | 327 | 256 | 291 | 296 | 276 |
| dehydrogenase | (IU/L) | | | | | | | | |
| (LDH) | | | | | | | | | |

All patents and other references cited in the specification are indicative of the level of skill of those skilled in the art to which the disclosure pertains, and are incorporated by reference in their entireties, including any tables and figures, to the same extent as if each reference had been incorporated by reference in its entirety individually.

One skilled in the art would readily appreciate that the present disclosure is well adapted to obtain the ends and advantages mentioned, as well as those inherent therein. The methods, variances, and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

What is claimed is:

1. A method for treating sickle cell disease in a subject in need thereof, comprising administering to the subject a first dose of Compound I of formula: 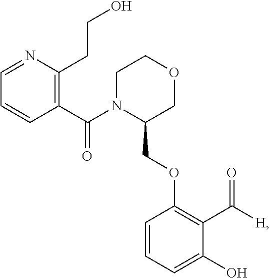
or a pharmaceutically acceptable salt thereof, for eight or more days,
followed thereafter by a second dose of Compound I, or a pharmaceutically acceptable salt thereof, wherein the administrations of the first dose and the second dose provide about 25% to about 60% hemoglobin occupancy by Compound I.

2. The method of claim 1, wherein the administrations of the first dose and the second dose provide about 30% to about 50% hemoglobin occupancy by Compound I.

3. A method for treating sickle cell disease in a subject in need thereof, comprising administering to the subject a first dose of about 200 mg per day to about 1600 mg per day of Compound I of formula: 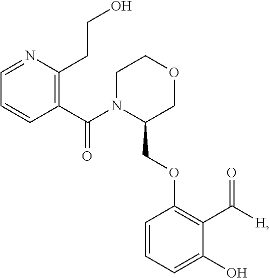
or a pharmaceutically acceptable salt thereof, for eight or more days,
followed thereafter by a second dose of about 25 mg per day to about 500 mg per day of Compound I, or a pharmaceutically acceptable salt thereof.

4. The method of claim 3, comprising administering to the subject a first dose of about 200 mg per day to about 600 mg per day of Compound I of formula: 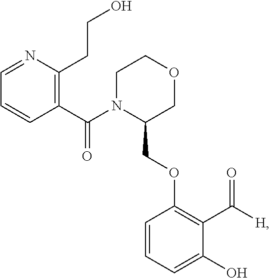
or a pharmaceutically acceptable salt thereof, for eight or more days,
followed thereafter by a second dose of about 25 mg per day to about 250 mg per day of Compound I, or a pharmaceutically acceptable salt thereof.

5. The method of any one of claims 1-4, wherein the first dose is administered for eight, nine, ten, eleven, twelve, thirteen, or fourteen days.

6. The method of any one of claims 1-4, wherein the first dose is administered for fifteen or more days.

7. The method of any one of claims 1-5, wherein the first dose is administered for fourteen days.

8. The method of any one of claims 1-7, wherein the second dose is about 25 mg per day to about 200 mg per day of Compound I, or a pharmaceutically acceptable salt thereof.

9. The method of any one of claims 1-8, wherein the second dose is about 200 mg per day of Compound I, or a pharmaceutically acceptable salt thereof.

10. The method of any one of claims 1-9, wherein the first dose is:
about 300 mg per day of Compound I, or a pharmaceutically acceptable salt thereof, administered for eight or more days, followed by a second dose of about 200 mg per day of Compound I, or a pharmaceutically acceptable salt thereof.

11. The method of any one of claims 1-10, wherein the first dose of Compound I, or a pharmaceutically acceptable salt thereof, is administered once daily (QD).

12. A method for treating sickle cell disease in a subject in need thereof, comprising administering to the subject a first dose of about 300 mg once daily (QD) of Compound I of formula: 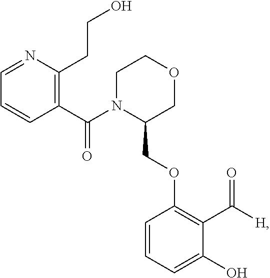
or a pharmaceutically acceptable salt thereof, for fourteen days,
followed thereafter by a second dose of 200 mg once daily (QD) of Compound I, or a pharmaceutically acceptable salt thereof.

13. The method of any one of claims 1-12, wherein the second dose of Compound I, or a pharmaceutically acceptable salt thereof, is administered for at least one week.

14. The method of any one of claims 1-13, wherein the second dose of Compound I, or a pharmaceutically acceptable salt thereof, is administered for at least ten weeks.

15. The method of any one of claims 1-13, wherein the second dose of Compound I, or a pharmaceutically acceptable salt thereof, is administered for at least fifty weeks.

16. The method of any one of claims 1-15, wherein the second dose of Compound I, or a pharmaceutically acceptable salt thereof, is administered for at least one year.

17. The method of any one of claims 1-16, wherein the second dose of Compound I, or a pharmaceutically acceptable salt thereof, is administered for at least five years.

18. The method of any one of claims 1-17, wherein the second dose of Compound I, or a pharmaceutically acceptable salt thereof, is administered for the lifetime of the subject.

19. The method of any one of claims 1-14, wherein the second dose of Compound I, or a pharmaceutically acceptable salt thereof, is administered for twelve weeks.

20. The method of any one of claims 1-19, wherein Compound I, or a pharmaceutically acceptable salt thereof, is administered as a pharmaceutical composition.

21. The method of any one of claims 1-19, wherein Compound I is administered as a pharmaceutical composition.

22. The method of any one of claims 1-20, wherein Compound I, or a pharmaceutically acceptable salt thereof, is administered orally.

23. The method of any one of claims 1-22, further comprising administering an additional therapeutic agent.

24. The method of claim 23, wherein the additional therapeutic agent is hydroxyurea.

25. The method of any one of claims 1-24, wherein the percent hemoglobin occupancy in the subject, reaches about 25% to about 60% after about 8 hours to about 24 hours following the completion of the first dose of Compound I, or a pharmaceutically acceptable salt thereof.

26. The method of claim 25, wherein the percent hemoglobin occupancy in the subject reaches about 25% to about 60% after about 8 hours to about 12 hours following the completion of the first dose of Compound I, or a pharmaceutically acceptable salt thereof.

27. The method of any one of claims 1-24, wherein the percent hemoglobin occupancy in the subject, reaches about 25% to about 60% prior to administration of the second dose of Compound I, or a pharmaceutically acceptable salt thereof.

28. The method of any one of claims 1-27, wherein the administration of Compound I, or a pharmaceutically acceptable salt thereof, provides a blood concentration of Compound I of about 50 μg/mL to about 800 μg/mL in the subject.

29. The method of claim 28, wherein the administration of Compound I, or a pharmaceutically acceptable salt thereof, provides a blood concentration of Compound I of about 300 μg/mL to about 475 μg/mL in the subject.

30. A compound for use in a method of treating sickle cell disease in a subject in need thereof, wherein the compound is Compound I of formula: 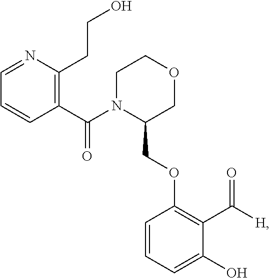
or a pharmaceutically acceptable salt thereof, and wherein the method is the method of any one of claims 1-29.

31. A compound for use in a method of treating sickle cell disease in a subject in need thereof, wherein the compound is Compound I of formula: 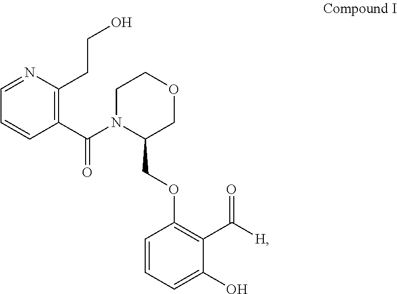
or a pharmaceutically acceptable salt thereof, and wherein the method comprises administering to the subject a first dose of Compound I, or a pharmaceutically acceptable salt thereof, for eight or more days,
followed thereafter by a second dose of Compound I, or a pharmaceutically acceptable salt thereof, wherein the administrations of the first dose and the second dose provide about 25% to about 60% hemoglobin occupancy by Compound I.

32. A compound for use in a method of treating, as claimed in claim 31, wherein the administration of the first dose and the second dose of Compound I provide about 30% to about 50% hemoglobin occupancy by Compound I.

33. A compound for use in a method of treating sickle cell disease in a subject in need thereof, wherein the compound is Compound I of formula: 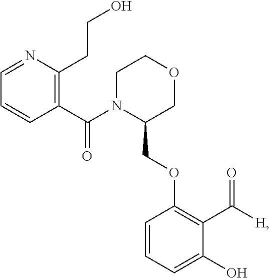
or a pharmaceutically acceptable salt thereof, and wherein the method comprises administering to the subject a first dose of about 200 mg per day to about 1600 mg pe day of Compound I, or a pharmaceutically acceptable salt thereof, for eight or more days,
followed thereafter by a second dose of about 25 mg per day to about 500 mg per day of Compound I, or a pharmaceutically acceptable salt thereof.

34. A compound for use in a method of treating, as claimed in any one of claims 31-33, wherein the method comprises administering to the subject a first dose of about 300 mg per day to about 1500 mg per day of Compound I, or a pharmaceutically acceptable salt thereof, for eight or more days, followed thereafter by a second dose of about 25 mg per day to about 250 mg per day of Compound I, or a pharmaceutically acceptable salt thereof.

35. A compound for use in a method of treating sickle cell disease in a subject in need thereof, wherein the compound is Compound I of formula: 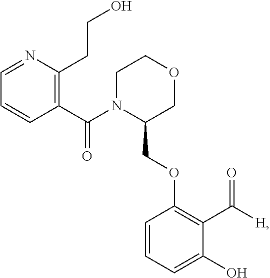
or a pharmaceutically acceptable salt thereof, and wherein the method comprises administering to the subject a first dose of about 300 mg once daily (QD) of Compound I, or a pharmaceutically acceptable salt thereof, for fourteen days,
followed thereafter by a second dose of 200 mg once daily (QD) of Compound I, or a pharmaceutically acceptable salt thereof.
